(19) United States
(12) Patent Application Publication
(10) Pub. No.: US 2024/0350754 A1
(43) Pub. Date: Oct. 24, 2024

(54) SCANNING AND IMAGING SYSTEM

(71) Applicant: WUHAN UNITED IMAGING LIFE SCIENCE INSTRUMENT CO., LTD., Wuhan (CN)

(72) Inventors: Hongfeng LIANG, Wuhan (CN); Feng WANG, Wuhan (CN); Yao XING, Shanghai (CN); Hailiang KE, Wuhan (CN)

(73) Assignee: WUHAN UNITED IMAGING LIFE SCIENCE INSTRUMENT CO., LTD., Wuhan (CN)

(21) Appl. No.: 18/755,611

(22) Filed: Jun. 26, 2024

Related U.S. Application Data
(63) Continuation of application No. PCT/CN2022/140031, filed on Dec. 19, 2022.

(30) Foreign Application Priority Data
Dec. 26, 2021 (CN) 202111606300.0
Dec. 26, 2021 (CN) 202111606311.9
May 20, 2022 (CN) 202210548607.8
May 20, 2022 (CN) 202221266069.5

(51) Int. Cl. A61M 16/01; A61M 16/00

(52) U.S. Cl. CPC A61M 16/01; A61M 16/0003; A61M 16/0051; A61M 16/024; A61M 2202/0007; A61M 2202/0241; A61M 2205/103; A61M 2205/18; A61M 2205/3303; A61M 2205/3368; A61M 2205/3561; A61M 2205/3576; A61M 2205/3653; A61M 2205/366; A61M 2205/502; A61M 2230/005; A61M 2230/06; A61M 2230/20; A61M 2230/40; A61M 2230/50; A61M 2250/00

(57) ABSTRACT

The embodiments of the present disclosure provide an imaging system, including: a scanning cabin management module configured to manage at least one scanning cabin, wherein each of the at least one scanning cabin is configured to place a subject; an imaging module configured to perform a scanning on the subject in the scanning cabin; and a control module configured to control the scanning cabin management module and/or the imaging module.

### CROSS-REFERENCE TO RELATED APPLICATIONS

This application is a Continuation of International Application No. PCT/CN2022/140031, filed on Dec. 19, 2022, which claims priority to Chinese Application No. 202210548607.8 and Chinese Application No. 202221266069.5, filed on May 20, 2022, and Chinese Application No. 202111606311.9 and Chinese Application No. 202111606300.0, filed on Dec. 26, 2021, the whole of each of which is incorporated by citations incorporated herein by reference in their entirety.

### TECHNICAL FIELD

The present disclosure relates to the technical field of life science instrumentation, and in particular to an imaging system.

### BACKGROUND

When performing life science research, animal experiments are often conducted, and an imaging device will be configured to perform imaging of the animal to clearly and realistically display anatomical structures, facilitate better observation of organ size, shape, and location, and display structures such as blood vessels and nerve fibers. In performing imaging of an animal using an imaging device (e.g., CT, SPECT, PET, MR, and any combination of imaging devices for each modality), the animal needs to be anesthesia or injected with a medicine, and the anesthesia animal needs to be kept warm.

The current animal imaging devices do not link the anesthesia device, holding device, and injection device together, requiring the experimenter to manually set the anesthesia concentration and heat preservation temperature before scanning the animal, manually injecting the medicine and manually filling in the injection information (medicine information, injection time, and injection dosage) into the experimental registry, resulting in a complex experimental process. Moreover, during the whole experimental process, the experimenter needs to stay in the lab for a long time (e.g., more than 10 hours) waiting for the experiment to be completed, which is time-consuming and laborious.

### SUMMARY

According to one of the embodiments of the present disclosure, an imaging system is provided. The imaging system may include a scanning cabin management module configured to manage at least one scanning cabin, each of the at least one scanning cabin is configured to place a subject; an imaging module configured to perform a scanning on the subject in the scanning cabin; and a control module configured to control the scanning cabin management module and/or the imaging module.

In some embodiments, the system further may include a function assistance module; the function assistance module may include at least one of: an anesthesia device, a temperature control device, or a physiological monitoring device; the anesthesia device may be configured to receive a first control signal sent by the control module, output an anesthesia gas to the scanning cabin based on the first control signal, and feed an anesthesia signal back to the control module; the temperature control device may be configured to receive a second control signal sent by the control module, adjust a temperature of the scanning cabin based on the second control signal, and feed a temperature signal back to the control module; and the physiological monitoring device may be configured to perform a physiological monitoring on the subject and feed a physiological parameter back to the control module.

In some embodiments, the control module may be configured to perform a signal transmission and instruction control on at least one of the scanning cabin management module, the imaging module, or the function assistance module through a wireless communication connection.

In some embodiments, the function assistance module further may include an injection device configured to receive a third control signal sent by the control device, inject the subject based on the third control signal, and feed an injection signal back to the control module; the control module may be connected to at least one of the anesthesia device, the temperature control device, the injection device, or the physiological monitoring device; the control module may be configured to control at least one of the anesthesia device, the temperature control device, the injection device, or the physiological monitoring device to perform a corresponding operation; and/or, to control the imaging module to perform a scanning based on detection information of at least one of the anesthesia device, the temperature control device, the injection device, or the physiological monitoring device.

In some embodiments, the system further may include a transfer module configured to transfer the scanning cabin from the scanning cabin management module to the imaging module.

In some embodiments, the control module may be connected to a transfer module; and the control module may be configured to control the transfer module to perform a corresponding operation.

In some embodiments, the scanning cabin management module may be provided with a plurality of cabin seats configured to place a plurality of scanning cabins; the transfer module may be provided between the scanning cabin management module and the imaging module, and may be configured to transfer the scanning cabin from the scanning cabin management module to the imaging module.

In some embodiments, the scanning cabin management module may include a mounting base and a rotating substrate, the rotating substrate may be rotatably mounted to the mounting base, and the plurality of cabin seats may be disposed on the rotating substrate.

In some embodiments, the scanning cabin management module may further include an adapter, the scanning cabin may be rotatably mounted to the rotating substrate through the adapter relative to the rotating substrate.

In some embodiments, the scanning cabin management module further may include a display screen configured to display a physiological parameter of the subject, the plurality of cabin seats may be distributed on the rotating substrate along a circumferential direction of the display screen.

In some embodiments, the system may further include a connector that may be mounted to the imaging module; and the transfer module may be capable of mounting the scanning cabin to the imaging module through the connector.

In some embodiments, the transfer module may include a displacement unit and a mechanical arm unit, the displacement unit may be able to drive the mechanical arm unit to perform a translation; and one end of the mechanical arm unit may be connected to the displacement unit and the other end of the mechanical arm unit may be configured to clamp the scanning cabin.

In some embodiments, the mechanical arm unit may include a clamping assembly and a rotating arm assembly, one end of the rotating arm assembly may be connected to the clamping assembly and the other end of the rotating arm assembly may be rotatably connected to the displacement unit; the rotating arm assembly may include a connecting arm and a first rotating motor, the first rotating motor may be mounted to the displacement unit, the connecting arm may connect the first rotating motor and the clamping assembly, and the first rotating motor may be capable of driving the connecting arm to rotate; and the clamping assembly may include a driving component and a pair of clamping jaws oppositely disposed, the driving component may connect the pair of clamping jaws and the rotating arm assembly, the driving component may be configured to drive the pair of clamping jaws to rotate relative to the rotating arm assembly, and drive the pair of clamping jaws to be clamped or opened.

In some embodiments, the subject may include an animal, and the first control signal may include an anesthesia gas parameter; the anesthesia gas parameter may include at least one of a concentration parameter of an anesthesia gas, a dosage parameter of an anesthesia gas, or an output rate parameter of an anesthesia gas, and the anesthesia device may output an anesthesia gas to the scanning cabin based on the anesthesia gas parameter.

In some embodiments, the subject may include an animal, and the first control signal may include an anesthesia gas parameter; the anesthesia gas parameter may include at least one of a concentration parameter of an anesthesia gas, a dosage parameter of an anesthesia gas, or an output rate parameter of an anesthesia gas, and the anesthesia device may output an anesthesia gas to the scanning cabin based on the anesthesia gas parameter.

In some embodiments, the temperature control device may include a heating pad, the heating pad may be disposed within the scanning cabin; or, the temperature control device may include a heating pipeline, the heating pipeline may be connected to the scanning cabin and may transfer hot gas/hot water in the heating pipeline into the scanning cabin.

In some embodiments, the subject may include an animal, and the third control signal may include an injection parameter; and the injection parameter may include at least one of an injection dosage parameter, an injection speed parameter, or an injection time parameter, and the injection device may inject a drug to the animal in the scanning cabin based on the injection parameter.

In some embodiments, the system further may include a communication device configured be connected to a mobile device through a wireless communication, and/or, the communication device may be configured be connected to a local terminal device through a wired or wireless communication; and the communication device may be electrically connected to the scanning cabin management module, the imaging module, and the function assistance module, respectively.

In some embodiments, the physiological parameter may include at least one of a heart frequency, a respiratory signal, a body temperature signal, or a blood oxygen signal, and the subject may be an animal; the physiological monitoring device may be configured to: monitor the physiological parameter of the animal, compare the physiological parameter with a preset threshold of the physiological parameter, and when the physiological parameter exceeds the preset threshold of the physiological parameter, the physiological monitoring device may feed a physiological parameter monitoring signal back to the communication device, and the communication device may transmit the physiological parameter monitoring signal to the mobile device.

In some embodiments, the control module centralizedly may control the anesthesia device, the injection device, and the temperature control device.

In some embodiments, the control module may be further configured to compare the received physiological parameter with a preset threshold of the physiological parameter, and when the physiological parameter exceeds the preset threshold of the physiological parameter, the control module may send out an abnormal reminder signal.

### BRIEF DESCRIPTION OF THE DRAWING

The present disclosure will be further illustrated by way of exemplary embodiments, which will be described in detail through the accompanying drawings. These embodiments are not limiting, and in these embodiments, the same numbering indicates the same structure, wherein:

FIG. 1 is a schematic diagram illustrating an application scenario of an exemplary imaging system according to some embodiments of the present disclosure;

FIG. 2 is a schematic diagram illustrating an exemplary imaging system according to some embodiments of the present disclosure;

FIG. 3 is a schematic diagram illustrating an exemplary imaging system according to some embodiments of the present disclosure;

FIG. 4 is a schematic diagram illustrating an exemplary adapter and scanning cabin installation according to some embodiments of the present disclosure;

FIG. 5 is a schematic diagram illustrating an exemplary scanning cabin according to some embodiments of the present disclosure;

FIG. 6 is a schematic diagram illustrating an exemplary adapter according to some embodiments of the present disclosure;

FIG. 7 is a schematic diagram illustrating an exemplary transfer module according to some embodiments of the present disclosure;

FIG. 8 is a flowchart illustrating an exemplary imaging system according to some embodiments of the present disclosure;

FIG. 9 is a flowchart illustrating an exemplary imaging system according to another embodiment of the present disclosure.

In the figure, 100 is an imaging system, 110 is an imaging apparatus, 120 is a processing device, 130 is a terminal, 131 is a mobile device, 132 is a tablet computer, 133 is a laptop computer, 134 is a desktop computer, 140 is a storage device, 150 is a network, 200 is an imaging system, 210 is a scanning cabin management module, 220 is an imaging module, 230 is a control module, 240 is a function assistance module, 250 is a transit module, 310 is a scanning cabin management module, 311 is a cabin seat, 312 is a mounting base, 312-1 is an assembly cavity, 312-2 is a sampling port, 313 is a rotating substrate, 314 is an adapter, 314-1 is a docking hole, 314-2 is a second fluid transfer channel, 314-3 is a communication protrusion, 314-4 is a second electrical connection line, 314-5 is a communication protrusion, 315 is a display screen, 316 is a handle, 320 is an imaging module, 321 is a radiation source, 322 is a detector, 323 is a scanning base, 323-1 is a scanning cavity, 323-2 is a base plate, 323-3 is a bracket, 323-4 is a connecting plate, 323-5 is a support plate, 324 is a scanning mobile unit, 324-1 is a connector, 330 is a transfer module, 331 is a displacement unit, 331-1 is a linear driver, 331-2 is the mounting slider, 332 is the mechanical arm unit, 332-1 is the clamping assembly, 332-11 is a driving component, 332-12 is a second rotating motor, 332-13 is a clamping connector, 332-2 is a rotating arm assembly, 332-21 is a connecting arm, 332-22 is a first rotary motor, 340 is a connector, 341 is a docking connector, 341-1 is a first fluid transfer channel, 341-2 is a communication recess, 341-3 is a first electrical connection line, 341-4 is a conduction recess, 342 is a motorized locking structure, 800 is an imaging system, 810 is a control module, 820 is an imaging module, 830 is anesthesia device, the 840 is a temperature control device, 850 is an injection device, 900 is an imaging system, 910 is a communication device, 920 is a mobile device, 930 is a physiological monitoring device, 940 is a temperature control device, 950 is anesthesia device, and 960 is an experimenter.

### DETAILED DESCRIPTION

In order to illustrate the technical solutions related to the embodiments of the present disclosure, a brief introduction of the drawings referred to in the description of the embodiments is provided below. Obviously, the drawings described below are only some examples or embodiments of the present disclosure. Those skilled in the art, without further creative efforts, may apply the present disclosure to other similar scenarios according to these drawings. Unless apparent from the locale or otherwise stated, like reference numerals represent similar structures or operations throughout the several views of the drawings.

It will be understood that the term “system,” “device,” “unit,” and/or “module” used herein are one method to distinguish different components, elements, parts, sections, or assembly of different levels in ascending order. However, the terms may be displaced by another expression in response to determining that they achieve the same purpose.

As used in the disclosure and the appended claims, the singular forms “a,” “an,” “a kind of,” and/or “the” may include plural forms unless the content clearly indicates otherwise. In general, the terms “comprise,” “includes,” and/or “comprising,” “include,” “includes,” and/or “including,” merely prompt to include steps and elements that have been clearly identified, and these steps and elements do not constitute an exclusive listing. The methods or devices may also include other steps or elements.

The flowcharts used in the present disclosure illustrate operations that systems implement according to some embodiments in the present disclosure, the related description is configured to better understand the medical imaging control method and/or system. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

When performing imaging on an animal using an imaging device (e.g., CT, SPECT, PET, MR, and any combination of imaging devices for each modality), the animal needs to be anesthesia to avoid poor imaging and motion artifacts caused by the physical activity during the scanning process. After the animal enters an anesthesia state, the body temperature of the animal may gradually decrease, and prolonged hypothermia may lead to the death of the animal. Therefore, the animal imaging device needs to be configured with an anesthesia device and a heat preservation device to be configured together to maintain the animal in a suitable life state.

The current animal imaging devices are not linked with the anesthesia device and the heat preservation device, and an experimenter needs to manually set an anesthesia concentration and a heat preservation temperature in advance before performing an imaging on the animal, which is a complicated operation procedure. The anesthesia concentration and the heat preservation temperature may affect the heartbeat rate of the animal, thereby affecting the metabolic state of the animal. For SPECT and PET imaging devices, the metabolic state of the animal may affect an imaging result, so it is necessary to try to ensure that the anesthesia concentration and the heat preservation temperature are consistent when repeating the experiment.

When scanning the animal using a CT or MR imaging device, the animal needs to be injected with a contrast agent to increase the contrast degree in a specific part of the image. When scanning the animal using a SPECT or PET imaging device, the animal needs to be injected with a radionuclide medicine. After injecting the medicine into the animal, the experimenter may need to manually fill in injection information (medicine information, injection time, and injection dose) into experiment registration information, and need to artificially control the scanning after medicine injection, which makes the whole experimental process complicated.

In addition, when performing scanning on the living animal, it is often necessary to experience a long period to obtain an image or a result needed for the experiment. When conducting such long experiments (e.g., more than 10 hours), it is time-consuming and labor-intensive for the experimenter to stay in the lab for a long period waiting for the completion of the experiment.

The embodiment of the present disclosure provides an imaging system for scanning an animal. The imaging system may include a scanning cabin management module, an imaging module, a control module, a transfer module, and a function assistance module. A plurality of scanning cabins may be located in a plurality of cabin seats of the scanning cabin management module and a subject may be located in a scanning cabin. Before the scanning of the subject, perform anesthesia, heat preservation, and injection may be performed on the subject, which may ensure that the subject is in a state to be scanned. Therefore, it is convenient for a transfer module to transfer the scanning cabin where the subject is placed from the scanning cabin management module to the imaging module in time to perform a scanning. After the scanning cabin where the subject is placed has been transferred to the imaging module, operations such as anesthesia, heat preservation, injection, etc., may be performed on the subject, which greatly shortens a scanning preparation time for the scanning of the subject and improves the working efficiency of the scanning cabin management system. In some embodiments, the heat preservation, the injection, and the scanning process of the animal may be fully automated by linking the control module with the scanning cabin management module, the imaging module, and the function assistance module (e.g., an anesthesia device, a temperature control device, an injection device, or a physiological monitoring device), automatically synchronizing the anesthesia gas parameter data, temperature parameter data, injection parameter data, and physiological parameter data in an experimental process to experimental registration information, which reduces the complexity of an operation process, improves the repeatability and precision of the experiment, and is more friendly to the operator. In some embodiments, the experimenter may remotely control a scanning cabin through a mobile device during the experimental process by connecting the communication device with the mobile device through wireless communication, and the electrical connection between the communication device and the physiological monitoring device, the temperature control device, the anesthesia device, and the injection device, respectively, such that the experimenter is not necessary to stay in the lab to wait for a completion of the experiment, which increases an convenience of the experimental process.

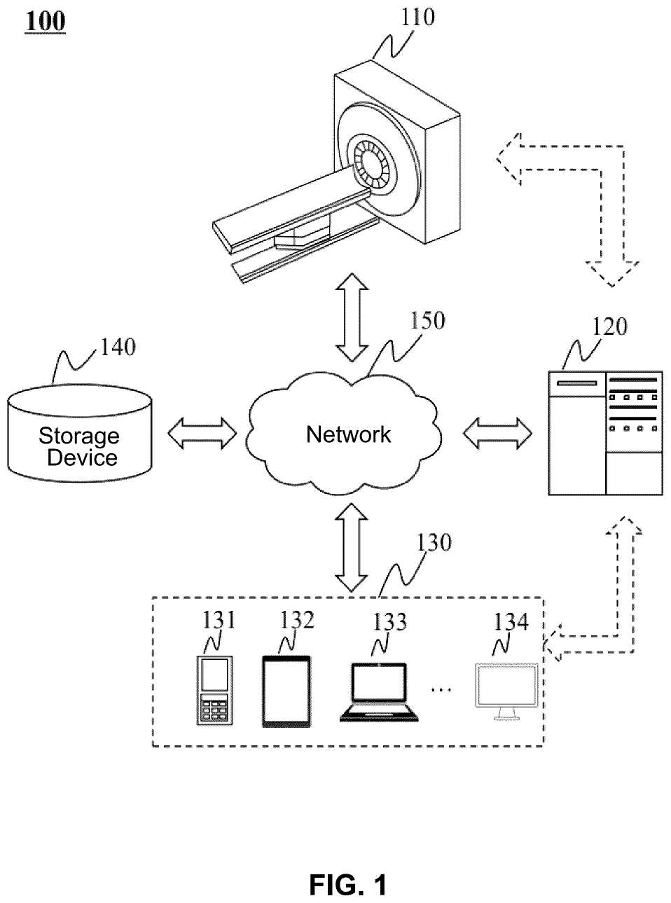

FIG. 1 is a schematic diagram illustrating an application scenario of an exemplary imaging system 100 according to some embodiments of the present disclosure.

As shown in FIG. 1, the imaging system 100 may include an imaging apparatus 110, a processing device 120, one or more terminals 130, a storage device 140, and a network 150. The components in the imaging system 100 may be connected in one or more of various manners. For example, as shown in FIG. 1, the imaging apparatus 110 may be connected to the processing device 120 through the network 150. As another example, the imaging apparatus 110 may be directly connected to the processing device 120, for example, the imaging apparatus 110 and the processing device 120 may be connected as indicated by dashed bi-directional arrows in FIG. 1. As another example, the storage device 140 may be connected directly to the processing device 120 (not shown in FIG. 1) or through the network 150. As a further example, the one or more terminals 130 may be connected directly to the processing device 120 (as indicated by the dashed bidirectional arrows connecting the one or more terminals 130 and the processing device 120) or through the network 150.

In some embodiments, the imaging apparatus 110 may include a scanning cabin management module, an imaging module, and a control module.

In some embodiments, the scanning cabin management module may be configured to manage at least one scanning cabin each of which is configured to place a subject. In some embodiments, the subject may include biological and/or non-biological subjects. In some embodiments, the subject may include the animal configured to be used exclusively for experiments, such as a mouse, a rat, a small pig, a rabbit, and a gopher. In some embodiments, the subject may include a specific portion of the animal, such as the head, the chest, the abdomen, etc., or a combination thereof. As another example, the subject may be an artificial component of animate or inanimate organic and/or inorganic material.

In some embodiments, the imaging module may be configured to perform an imaging on a subject within a scanning cabin to obtain scanning data (e.g., a scanning image, etc.) of the subject. In some embodiments, the imaging module may include a non-invasive bioimaging device that is configured to be used for disease diagnosis or research purposes. For example, the imaging module may include a single-modality scanner and/or a multi-modality scanner. The single-modality scanner may include, such as an ultrasound scanner, an X-ray scanner, a computed tomography (CT) scanner, a magnetic resonance imaging (MRI) scanner, a positron emission tomography (PET) scanner, an optical coherence tomography (OCT) scanner, an ultrasound (US) scanner, an intravascular ultrasound (IVUS) scanner, near-infrared spectroscopy (NIRS) scanners, a far infrared (FIR) scanner, and the like. The multi-modality scanner may include, such as an X-ray imaging-magnetic resonance imaging (X-ray-MRI) scanner, a positron emission tomography-X-ray imaging (PET-X-ray) scanner, a single photon emission computed tomography-magnetic resonance imaging (SPECT-MRI) scanner, a single photon emission computed tomography-computed tomography (SPECT-CT) scanner, a positron emission tomography-computed tomography (PET-CT) scanner, a digital subtraction angiography-magnetic resonance imaging (DSA-MRI) scanner, and the like. The scanners provided above are merely provided for the purpose of illustration and are not intended to limit the scope of the present disclosure. As used herein, the term “imaging modality” or “modality” refers to an imaging method or technique that collects, generates, processes, and/or analyzes imaging information of a target subject.

In some embodiments, the control module may be configured to control the scanning cabin management module and/or the imaging module. In some embodiments, the control module may be a portion of the processing device 120.

In some embodiments, the data obtained by the imaging apparatus 110 (e.g., the scanning image or the monitoring information, etc.) may be transmitted to the processing device 120 for further analysis. Additionally, or alternatively, the data obtained by the imaging apparatus 110 may be sent to a terminal device (e.g., the one or more terminals 130) for display and/or a storage device (e.g., the storage device 140) for storage.

The processing device 120 may process data and/or information obtained and/or extracted from the imaging apparatus 110, the one or more terminals 130, the storage device 140, and/or other storage devices. For example, the processing device 120 may obtain the monitoring information (e.g., a temperature, a concentration of the anesthesia gas in the scanning cabin) from the imaging apparatus 110. In some embodiments, the processing device 120 may control the imaging apparatus 110. For example, the processing device 120 may control the scanning cabin management module to operate and to perform the anesthesia on the subject or to adjust the temperature in the scanning cabin. As another example, the processing device 120 may control the imaging module to perform scanning on the subject.

In some embodiments, the processing device 120 may be a single server or a server group. The server group may be centralized or distributed. In some embodiments, the processing device 120 may be local or remote. In some embodiments, the processing device 120 may be implemented on a cloud platform. Merely by way of example, a cloud platform may include a private cloud, a public cloud, a hybrid cloud, a community cloud, a distributed cloud, an on-premises cloud, a multi-tiered cloud, etc., or any combination thereof.

In some embodiments, the processing device 120 may be implemented on a computing device. In some embodiments, the processing device 120 may be implemented on the terminal (e.g., the one or more terminals 130). In some embodiments, the processing device 120 may be implemented on the imaging device (e.g., the imaging module 220). For example, the processing device 120 may be integrated into the one or more terminals 130 and/or the imaging apparatus 110.

The one or more terminals 130 may be connected to the imaging apparatus 110 and/or the processing device 120, and be configured to input/output the information and/or data. For example, a user may interact with the scanning system 110 through the one or more terminals 130 to control one or more components of imaging apparatus 110 (e.g., inputting animal information, etc.). As another example, the imaging apparatus 110 may output generated scanning images or monitoring information to the one or more terminals 130 for presentation to the user.

In some embodiments, the one or more terminals 130 may include a mobile device 131, a tablet 132, a laptop computer 133, a desktop computer 134, etc., or any combination thereof. In some embodiments, the mobile device 131 may include a smart home device, a wearable device, a smart mobile device, a virtual reality device, an augmented reality device, etc., or any combination thereof. In some embodiments, the one or more of the terminal 130 may remotely operate the imaging apparatus 110. In some embodiments, the one or more terminals 130 may operate the imaging apparatus 110 through a wireless connection. In some embodiments, one or more terminals 130 may be a portion of the processing device 120. In some embodiments, the one or more terminals 130 may be omitted.

The storage device 140 may store data and/or instruction. In some embodiments, the storage device 140 may store data obtained from the one or more terminals 130 and/or the processing device 120. For example, the storage device 140 may store a scanning image, an anesthetic parameter, a temperature parameter, an injection parameter, a physiological parameter, and the like. In some embodiments, the storage device 140 may store the data and/or instruction that the processing device 120 may execute or use to perform the exemplary process described in the present disclosure.

In some embodiments, the storage device 140 may include a mass storage device, a removable storage device, volatile read-write memory, read-only memory (ROM), and the like, or any combination thereof. An exemplary mass storage may include a disk, an optical disk, a solid state drive, and the like. Exemplary removable storage may include a flash drive, a floppy disk, an optical disk, a memory card, a zip disk, a magnetic tape, and the like. An exemplary volatile read/write memory may include a random access memory (RAM). In some embodiments, the storage device 140 may be implemented on a cloud platform. In some embodiments, the storage device 140 may be a portion of the processing device 120.

The network 150 may include any suitable network that may facilitate the exchange of information and/or data for the imaging system 100. In some embodiments, one or more components of the scanning control system 100 (e.g., the imaging apparatus 110, the one or more terminals 130, the processing device 120, or the storage device 140) may be in communication with one or more other components of the scanning control system 100 to transmit information and/or data. In some embodiments, the network 150 may be any type of wired or wireless network or a combination thereof. For example, the network 150 may be and/or include a public network (e.g., the Internet), a private network (e.g., a local area network (LAN), a wide area network (WAN), etc.), a wired network (e.g., an Ethernet), a wireless network (e.g., an 802.11 network, a Wi-Fi network, etc.), a cellular network (e.g., a long term evolution (LTE) network), a frame relay network, a virtual private network (“VPN”), a satellite network, a telephone network, a router, a hub, a switch, a server computer, and/or any combination thereof. In some embodiments, the network 150 may include one or more network access points.

It should be noted that the above description of the imaging system is merely provided for the purpose of illustrating and is not intended to limit the scope of the present disclosure. Various variations and amendments may be made according to the present disclosure for those skilled in the art. However, these variations and amendments do not depart from the scope of the present disclosure. For example, the imaging apparatus 110, the processing device 120, and the one or more terminals 130 may share a storage device 140 or may have their own storage devices.

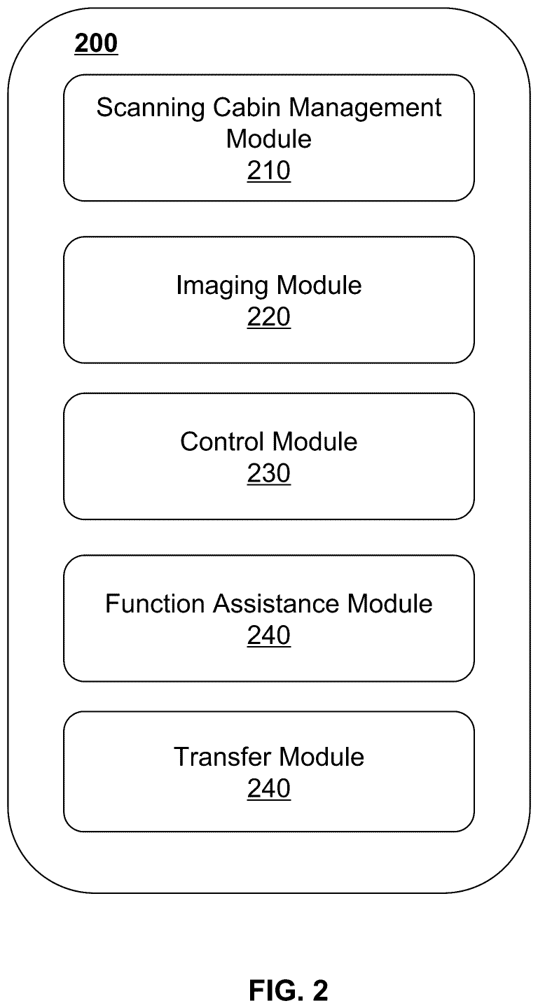

FIG. 2 is a schematic diagram illustrating an exemplary imaging system 200 according to some embodiments of the present disclosure.

As shown in FIG. 2, in some embodiments, the imaging system 200 may include a scanning cabin management module 210, an imaging module 220, a control module 230, a function assistance module 240, and a transfer module 250. It should be noted that modules, units, and sub-units described in the present disclosure may be realized through hardware, software, or a combination of software and hardware. The implementation of hardware may be realized by using a circuit or structure composed of physical components. The implementation of software may be realized by storing the operations corresponding to the modules, the units, and the sub-units in memory in the form of a code, which is executed by appropriate hardware such as a microprocessor. When the modules, the units, or the sub-units described in the present disclosure perform its operation, it may refer either to a software code containing the function being executed or to hardware having the function being used, in response to determining that not otherwise specified. At the same time, the modules, the units, and the sub-units mentioned herein do not limit the structure of their corresponding hardware when they correspond to the hardware, as long as the hardware that may realize their function is within the scope of protection of the present disclosure. For example, the different modules, units, and subunits mentioned herein may correspond to the same hardware structure. For example, the same module, unit, or sub-unit mentioned herein may correspond to a plurality of independent hardware structures.

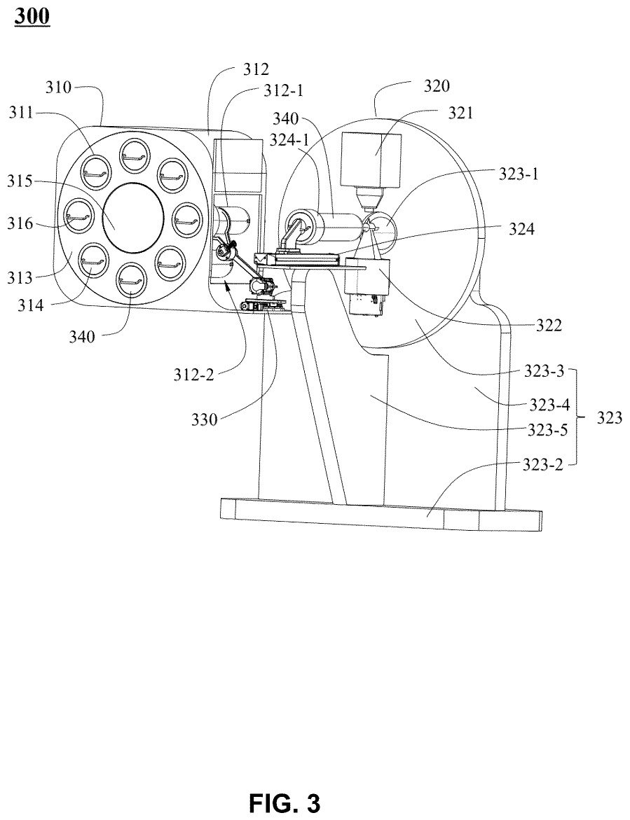

The scanning cabin management module 210 may be configured to manage at least one scanning cabin that is configured to place a subject. More descriptions of the scanning cabin management module 210 may be found in the descriptions in other parts of the present disclosure (e.g., FIG. 3), which will not be repeated herein.

The imaging module 220 may be configured to perform scanning on a subject within a scanning cabin. The imaging module 220 may include an imaging apparatus (e.g., the imaging apparatus 110). More descriptions of the imaging module 220 may be found in the descriptions in other parts of the present disclosure (e.g., FIG. 1 and FIG. 3), which will not be repeated herein.

The control module 230 may be configured to control the scanning cabin management module 210 and/or the imaging module 220.

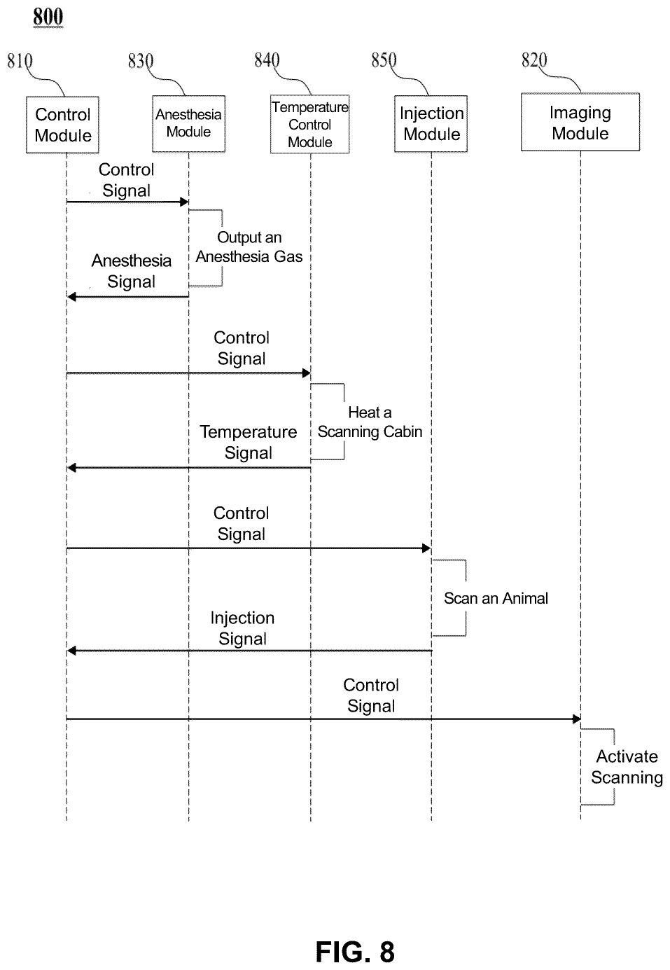

In some embodiments, the control module 230 may include a local terminal device, configured to perform a signal transmission and instruction control on at least one of the scanning cabin management module 210, the imaging module 220, the function assistance module 240, and the transfer module 250 through a wired communication connection. In some embodiments, the local terminal device may include a local desktop computer, such as the desktop computer 134 in FIG. 1. In some embodiments, the control module 230 may include a mobile device that is configured to perform the signal transmission and instruction control on at least one of the scanning cabin management module 210, the imaging module 220, the function assistance module 240, and the transfer module 250 through a wireless communication connection. In some embodiments, the control module 230 may include a local terminal device and a mobile device that are configured to perform the signal transmission and instruction control on at least one of the scanning cabin management module 210, the imaging module 220, the function assistance module 240, and the transit module 250 through the wired communication connection and the wireless communication connection, respectively. In some embodiments, the control module may also be a software module. In some embodiments, the software module may be configured on the local terminal device and/or the mobile device. In some embodiments, the software module may be stored in the storage device 140, and in some embodiments, the software module may run on the processing device 120. In some embodiments, the functions of the control module may be realized by hardware such as the local terminal device, the mobile device, the storage device, the processing device, and the like. More descriptions of the control module 230 may be found in the descriptions in other parts of the present disclosure (e.g., FIG. 8) and will not be repeated herein.

In some embodiments, the function assistance module 240 may include at least one of an anesthesia device, a temperature control device, or a physiological monitoring device. For example, the function assistance module 240 may include the anesthesia device, the temperature control device, and the physiological monitoring device. In some embodiments, the function assistance module 240 may further include an injection device. In some embodiments, the anesthesia device may be configured to receive a first control signal sent by the control module 230, output an anesthesia gas to the scanning cabin based on the first control signal, and feed an anesthesia signal back to the control module 230. In some embodiments, the first control signal may include a target or desired value or range of the anesthesia gas parameter (also referred to as a target anesthesia gas parameter, e.g., an anesthesia parameter threshold), such as the concentration of the anesthesia gas, the dosage of the anesthesia gas, etc. In some embodiments, the temperature control device may be configured to receive a second control signal sent by the control module 230, adjust the actual temperature of the scanning cabin based on the second control signal, and feed a temperature signal back to the control module 230. In some embodiments, the second control signal may include a target or desired value or range of the temperature (also referred to as a target temperature, e.g., a temperature parameter threshold), such as the target body temperature, the target temperature in the scanning cabin, etc. In some embodiments, the injection device may be configured to receive a third control signal sent by the control module 230, perform an injection on the subject based on the third control signal, and feed an injection signal back to the control module 230. In some embodiments, the third control signal may include a target or desired value or range of the injection parameter (also referred to as a target injection parameter, e.g., the injection speed threshold), such as the injection speed, etc. In some embodiments, the physiological monitoring device may be configured to receive a fourth control signal sent by the control module 230, perform physiological monitoring on the subject based on the fourth control signal, and feed a physiological parameter signal back to the control module 230. In some embodiments, the fourth control signal may include a target or desired value or range of the physiological parameter (also referred to as a target physiological parameter, e.g., the heartbeat rate threshold), such as the heartbeat rate, etc. In some embodiments, at least a portion of the function assistance module 240 may be provided in the scanning cabin, and at least another portion of the function assistance module 240 may be provided in the scanning cabin management module 210 and the imaging module 220. For example, the function assistance module 240 may include a volatile tank and an anesthesia detection assembly, the volatile tank may store the anesthesia gas, the volatile tank may be disposed on the scanning cabin management module 210 and the imaging module 220, and the anesthesia detection assembly may be disposed within the scanning cabin. More descriptions of the function assistance module 240 may be found in the descriptions in other parts of the present disclosure (e.g., FIG. 3-FIG. 9), which will not be repeated herein.

Further, in some embodiments, the control module 230 may be connected to at least one of the anesthesia device, the temperature control device, the injection device, the physiological monitoring device, or the transfer module 250. In some embodiments, the control module 230 may be configured to control at least one of the anesthesia device, the temperature control device, the injection device, the physiological monitoring device, or the transfer module 250 to perform a corresponding operation. In some embodiments, the control module 230 may control the imaging module 220 to perform a scanning based on detection information of at least one of the anesthesia device, the temperature control device, the injection device, or the physiological monitoring device. In some embodiments, the control module 230 may be connected to the scanning cabin management module 210 to control the at least one of the anesthesia device, the temperature control device, the injection device, or the physiological monitoring device to perform a corresponding operation through the scanning cabin management module 210. In some embodiments, the control module 230 may be connected to the imaging module 220 to control the imaging module 220 to perform a scanning based on the detection information of at least one of the anesthesia device, the temperature control device, the injection device, or the physiological monitoring device.

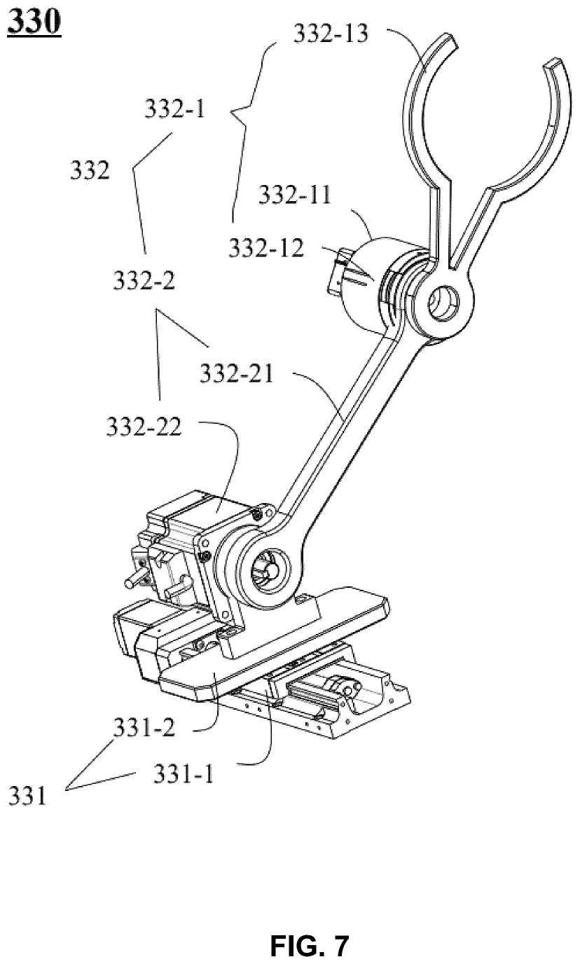

The transfer module 250 may be configured to transfer a scanning cabin from the scanning cabin management module 210 to the imaging module 220. More descriptions of the transfer module 250 may be found in the descriptions in other parts of the present disclosure (e.g., FIGS. 3 and 7), which will not be repeated herein.

It should be noted that the above description of the imaging system 200 and the modules thereof is provided for the purpose of descriptive convenience and does not limit the present disclosure to the scope of the cited embodiments. It should be understood that for those skilled in the art, after understanding the principle of the system, it may be possible to make any combination of the individual modules or form a sub-system to connect with other modules without departing from this principle. However, such amendments and variations remain within the scope of the present disclosure.

In some embodiments, the imaging system may include a scanning cabin management module, an imaging module, and a control module. In some embodiments, the imaging system may be configured to automatically control and/or remotely control an entire imaging process (e.g., a scanning preparation process, a state to be scanned, a transit process, or a scanning process) of the subject, to achieve repeatability and control accuracy, and to improve convenience, in the scanning process or the experimental process.

In some embodiments, the scanning cabin management module may include at least one cabin seat each of which is configured to place a scanning cabin, and the scanning cabin management module may be configured to manage the at least one scanning cabin. In some embodiments, the scanning cabin may be configured to place a subject. In some embodiments, the at least one cabin seat of the scanning cabin management module may be rotated to drive the rotation of the at least one scanning cabin to rotate the at least one scanning cabin to a specified position, such as a position near the imaging module. More descriptions of the scanning cabin management module may be found in the descriptions in other parts of the present disclosure (e.g., FIG. 3), which will not be repeated herein.

In some embodiments, the imaging module may be configured to perform a scanning on the subject within a scanning cabin. In some embodiments, the imaging module may be configured to perform the scanning on the subject within the scanning cabin that is transferred to the imaging module. In some embodiments, the imaging module may perform the scanning on the subject inside each of the at least one scanning cabin in sequence to improve the continuity and convenience of a plurality of successive scanning processes. More descriptions of the imaging module may be found in the descriptions in other parts of the present disclosure (e.g., FIG. 3), which will not be repeated herein.

In some embodiments, the control module may be configured to control the scanning cabin management module and/or the imaging module. In some embodiments, the control module may be configured to control the at least one cabin seat of the scanning cabin management module to rotate. In some embodiments, the control module may be configured to control the imaging module to perform the scanning on a subject that is transferred to the imaging module through the rotation of the at least one cabin seat. More descriptions of the control module may be found in the descriptions in other parts of the present disclosure (e.g., FIG. 8) and will not be repeated herein.

In some embodiments, the imaging system may further include a function assistance module and a transfer module.

In some embodiments, the function assistance module may be a module configured to perform an assistance operation (e.g., anesthesia, temperature control, injection, physiological monitoring, etc.). In some embodiments, the function assistance module may include at least one of the anesthesia device, the temperature control device, or the physiological monitoring device. In some embodiments, the function assistance module may perform a corresponding assistance operation based on a control signal of the control module. In some embodiments, the control signal of the control module may include at least one of an anesthesia control instruction, a temperature control instruction, or a physiological monitoring instruction. In some embodiments, the assistance operation may include at least one of inputting anesthesia gas into the scanning cabin, adjusting the temperature of the scanning cabin or the temperature of the subject, performing a physiological monitoring of the subject, or other operations. In some embodiments, the function assistance module may further include an injection device. In some embodiments, the control signal of the control module may further include an injection control instruction. In some embodiments, the assistance operation may include injecting the subject inside the scanning cabin.

In some embodiments, the anesthesia device may be configured to receive the first control signal sent by the control module and output the anesthesia gas to the scanning cabin based on the first control signal, and feed the anesthesia signal back to the control module. In some embodiments, the anesthesia device may be configured to receive the anesthesia control instruction from the control module, and output the anesthesia gas to the scanning cabin based on the anesthesia control instruction, and feed the anesthesia signal (e.g., concentration of the anesthesia gas and/or dosage of the anesthesia gas) back to the control module. In some embodiments, the control module sends the anesthesia control instruction to the scanning cabin management module or the imaging module, and the anesthesia device may be configured to receive a corresponding anesthesia control instruction from the scanning cabin management module or the imaging module, and output the anesthesia gas to the scanning cabin based on the corresponding anesthesia control instruction, and feed the anesthesia signal back to the scanning cabin management module or the imaging module, and the scanning cabin management module or the imaging module may feed the anesthesia signal back to the scanning cabin management module or the imaging module, and the scanning cabin management module or the imaging module may feed the received anesthesia signal back to the control module.

In some embodiments, the temperature control device may be configured to receive the second control signal sent by the control module, and adjust the temperature of the scanning cabin based on the second control signal, and feed the temperature signal back to the control module. In some embodiments, the temperature control device may be configured to receive the temperature control instruction from the control module, adjust the temperature of the scanning cabin based on the temperature control instruction, and feed the temperature signal back to the control module. In some embodiments, the temperature control device may be configured to receive the temperature control instruction sent by the control module, and adjust the body temperature of the subject based on the temperature control instruction, and feed a body temperature parameter (i.e., the actual body temperature after adjusting the temperature of the scanning cabin) back to the control module. In some embodiments, the control module may send the temperature control instruction to the scanning cabin management module or the imaging module, and the temperature control device may be configured to receive a corresponding temperature control instruction from the scanning cabin management module or the imaging module, and adjust temperature of the scanning cabin based on the received temperature control instruction, and feed the temperature parameter (i.e., the actual temperature in the scanning cabin after adjusting the temperature of the scanning cabin) back to the scanning cabin management module or the imaging module, and the scanning cabin management module or the imaging module may feed the temperature signal back to the control module.

In some embodiments, the injection device may be configured to receive a third control signal sent by the control module, perform the injection on the subject based on the third control signal, and feed the injection signal back to the control module. In some embodiments, the injection device may be configured to receive an injection control instruction from the control module, perform the injection on the subject based on the injection control instruction, and feed the injection signal (e.g., an injection dosage and/or an actual injection speed) back to the control module. In some embodiments, the control module may send the injection control instruction to the scanning cabin management module or the imaging module, the injection device may be configured to receive a corresponding injection control instruction from the scanning cabin management module or the imaging module, and perform injection on the subject based on the received injection control instruction, and feed the injection signal back to the scanning cabin management module or the imaging module, and the scanning cabin management module or the imaging module may feed the corresponding injection signal back to the control module.

In some embodiments, the physiological monitoring device may be configured to receive a fourth control signal sent by the control module, perform physiological monitoring on the subject based on the fourth control signal, and feed the physiological parameter signal back to the control module. In some embodiments, the physiological monitoring device may be configured to receive a physiological monitoring instruction from the control module, perform physiological monitoring on the subject based on the physiological monitoring instruction, and feed the physiological parameter signal back to the control module. In some embodiments, the control module may send a physiological monitoring instruction to the scanning cabin management module or the imaging module, the physiological monitoring device may be configured to receive a corresponding physiological monitoring instruction from the scanning cabin management module or the imaging module, perform physiological monitoring on the subject based on the received physiological monitoring instruction, and feed the physiological parameter signal back to the scanning cabin management module or the imaging module, and the scanning cabin management module or the imaging module may feed the corresponding injection signal to the control module.

In some embodiments, the transfer module may perform a transfer operation based on a fifth control signal generated by the control module. In some embodiments, the fifth control signal generated by the control module may further include a transfer instruction. In some embodiments, the transfer module may receive the transfer instruction from the control module and transfer at least one scanning cabin from the scanning cabin management module to the imaging module, to perform the scanning based on the transfer instruction.

By placing the subject in the scanning cabin, then placing the plurality of scanning cabins in the plurality of cabin seats of the scanning cabin management module, and performing anesthesia, heat preservation, and/or injection operations on the subject before scanning, the subject may be guaranteed to be in the state to be scanned, and it is convenient for the transfer module to perform a timely transfer on the scanning cabin where the subject is placed from the scanning cabin management module to the imaging module, which greatly shortens the time for preparation of the scanning on the subject. Further, compared to a manual operation of handling the scanning cabin, the transfer of the scanning cabin using the transfer module greatly shortens the scanning preparation time of the subject and improves the work efficiency of the imaging system.

In some embodiments, the control module may include a local terminal device. In some embodiments, the local terminal device may include a local desktop computer. In some embodiments, the local terminal device may perform the signal transmission and instruction control on at least one of the scanning cabin management module, the imaging module, the function assistance module, and the transfer module through the wired communication connection. In some embodiments, the control module may include a mobile device. In some embodiments, the mobile device may include a cell phone. In some embodiments, the mobile device may perform the signal transmission and instruction control on the at least one of the scanning cabin management module, the imaging module, the function assistance module, and the transfer module through the wireless communication connection. In some embodiments, the wireless communication may include at least one of medium wave communication, short wave communication, ultra-short wave communication, microwave communication, or satellite communication. In some embodiments, the mobile device may send a control signal to the scanning cabin management module through the wireless communication connection to control the scanning cabin management module, e.g., to control at least one scanning cabin of the scanning cabin management module to rotate. In some embodiments, the mobile device may send a scanning signal to the imaging module through the wireless communication connection to control the imaging module to perform the scanning on the subject. In some embodiments, the mobile device may send the control signal to the function assistance module through the wireless communication connection to control the function assistance module to perform a corresponding assistance operation on the scanning cabin or the subject. In some embodiments, the mobile device may send a transfer signal to the transfer module through the wireless communication connection to control the transfer module to transfer the scanning cabin from the scanning cabin management module to the imaging module. More descriptions of the control process of the mobile device may be found in the relevant description of the control module, which will not be repeated herein. In some embodiments, the control module may include the local terminal device and a mobile device, and the local terminal device and the mobile device may perform the signal transmission and instruction control on the at least one of the scanning cabin management module, the imaging module, the function assistance module, and the transit module, respectively, through the wired communication connection and the wireless communication connection.

The mobile device may be connected to other modules in the imaging system through wireless communication, which realizes a remote control and monitoring of the imaging system by the mobile device and does not need to stay in the lab for a long time to wait for the completion of the experiment, which enhances the convenience of the whole experimental process.

In some embodiments, the control module may be connected to the at least one of the anesthesia device, the temperature control device, the injection device, the physiological monitoring device, or the transfer module. In some embodiments, the connection may be a communication connection through wireless communication or wired communication.

In some embodiments, the control module may be configured to control at least one of the anesthesia device, the temperature control device, the injection device, the physiological monitoring device, or the transfer module to perform a corresponding operation. In some embodiments, the control module may be configured to control the anesthesia device to input anesthesia gas into the scanning cabin. In some embodiments, the control module may be configured to control the temperature control device to adjust the target temperature of the scanning cabin or the target body temperature of the subject. In some embodiments, the control module may be configured to control the injection device to inject the subject within the scanning cabin. In some embodiments, the control module may be configured to control the physiological monitoring device to perform a physiological parameter monitoring on the subject in the scanning cabin, to obtain a monitoring physiological parameter. In some embodiments, the control module may be configured to control the transfer module to transfer the scanning cabin from the scanning cabin management module to the imaging module.

In some embodiments, the control module may be configured to control the imaging module to perform the scanning based on the detection information of at least one of the anesthesia device, the temperature control device, and the physiological monitoring device. In some embodiments, the control module may be configured to control the imaging module to perform the scanning based on the detection information of the injection device. In some embodiments, the control module may be configured to control the imaging module to perform the scanning based on the concentration of the anesthesia gas or the dosage of the anesthesia gas in the anesthesia device. In some embodiments, the control module may determine that the subject is in an anesthesia state in response to determining that the concentration of the anesthesia gas or dosage of the anesthesia gas has reached an anesthesia threshold, thereby controlling the imaging module to perform the scanning process. In some embodiments, the control module may determine that the subject is in a survival state based on the temperature in the scanning cabin or the body temperature of the subject, thereby controlling the imaging module to perform the scanning. In some embodiments, the control module may determine that the subject is in the state to be scanned based on the injection speed and injection dosage of the injection device, thereby controlling the imaging module to perform the scanning. In some embodiments, the control module may determine, based on the physiological parameter, that the subject is in the survival state or the state to be scanned, thereby controlling the imaging module to perform the scanning. More descriptions of the control module may be found in the descriptions in other parts of the present disclosure and will not be repeated herein.

By linking the control module with the imaging module, the anesthesia device, the temperature control device, the injection device, or the physiological monitoring device, the anesthesia, heat preservation, injection, and scanning of the animals are fully automated, and an intermediate process does not require or minimize the participation of operators, reducing the complexity of the operation process and improving the repeatability and precision of the experiment.

FIG. 3 is a schematic diagram illustrating an exemplary imaging system 300 according to some embodiments of the present disclosure.

As shown in FIG. 3, in some embodiments, an imaging system 300 may include a scanning cabin management module 310, an imaging module 320, and a transfer module 330. In some embodiments, the imaging system 300 may also include one or more scanning cabins 340 each of which is configured to place a subject. In some embodiments, the imaging system 300 may further include a control module (not shown in FIG. 3) configured to control the scanning cabin management module 310 and/or the imaging module 320.

In some embodiments, the scanning cabin management module 310 may be provided with a plurality of cabin seats 311 configured to place a plurality of scanning cabins 340. In some embodiments, the imaging module 320 may be configured to perform the scanning on the subject within a scanning cabin 340. In some embodiments, the transfer module 330 may be disposed between the scanning cabin management module 310 and the imaging module 320, and the transfer module 330 may be able to transfer the scanning cabin 340 from the scanning cabin management module 310 to the imaging module 320.

By placing a subject in a scanning cabin 340, and then placing the plurality of scanning cabins 340 on the plurality of cabin seats 311 of the scanning cabin management module 310, it may be ensured that the subject is in the state to be scanned (e.g., the anesthesia state), and it is convenient for the transfer module 330 to transfer the scanning cabin 340 where the subject is placed from the scanning cabin management module 310 to the imaging module 320 in a timely manner, thereby greatly shortening the scanning preparation time of the subject, improving the work efficiency of the imaging system. Moreover, relative to the operation of manual handling the scanning cabins, a transfer of the scanning cabin 340 where the subject is placed by using the transfer module 330 also shortens the transfer time of the scanning cabin 340, thereby effectively improving the work efficiency of the imaging system. Further, the transfer of the scanning cabin 340 where the subject is placed by using the transfer module 330 realizes an automated workflow of the imaging system. Further, relative to the operation of manual handling, the transfer of the scanning cabin 340 where the subject is placed by using the transfer module 330 effectively eliminates the hazard of radiation exposure to the staff.

In some embodiments, the imaging module 320 may be a CT device. In some embodiments, the CT device may be a micro CT (micro CT) device, and the micro CT device may be configured to image the animal or other biological tissue samples. Since the micro CT device is smaller in size, the micro CT device occupies less space, which greatly saves device land.

In some embodiments, as shown in FIG. 1, the imaging module 320 may include a radiation source 321, a detector 322, and a scanning base 323. In some embodiments, the scanning base 323 may be provided with a scanning cavity 323-1, and the radiation source 321 and the detector 322 may be disposed on different sides of the scanning cavity 323-1. In some embodiments, at least a portion of the scanning cabin 340 may extend into the scanning cavity 323-1 during the scanning. In some embodiments, the radiation source 321 may be configured to emit a radiation ray, and the detector 322 may be configured to receive the radiation ray, and the radiation source 321 and the detector 322 may cooperate with each other to realize scanning detection of the subject. In some embodiments, the imaging module 320 may be provided with an annular housing (not shown in FIG. 3), the scanning cavity 323-1 is provided in a center portion of the annular housing, at least a portion of the scanning cabin 340 may extend into the scanning cavity 323-1, and the radiation source 321 and the detector 322 may be provided in the annular housing.

In some embodiments, the scanning base 323 may include a base plate 323-2, a bracket 323-3, and a connecting plate 323-4 connecting the base plate 323-2 and the bracket 323-3, and the scanning base 323 may be disposed in a horizontal plane through the base plate 323-2. In some embodiments, both the radiation source 321 and the detector 322 may be mounted on the bracket 323-3. In some embodiments, the scanning cavity 323-1 may be disposed at the center portion of the bracket 323-3, and the scanning cavity 323-1 may be disposed at a preset height position (e.g., 0.8 m, 1 m, or 1.2 m) of the bracket 323-3 from the horizontal plane. In some embodiments, the bracket 323-3 may drive the radiation source 321 and the detector 322 to rotate around an axis of the bracket 323-3 to enable the radiation source 321 and the detector 322 to scan the subject from different angles.

In some embodiments, the scanning base 323 may include a support plate 323-5. In some embodiments, one end of the support plate 323-5 may connect the base plate 323-2 to the connecting plate 323-4, and the other end of the support plate 323-5 may extend in a direction away from the horizontal plane to be configured to support the transfer module 330.

Further, in some embodiments, as shown in FIG. 3, the imaging module 320 may include a scanning movement unit 324. In some embodiments, the scanning movement unit 324 may be disposed above the support plate 323-5. By providing the scanning moving unit 324, the scanning moving unit 324 may be activated to drive the scanning cabin 340 to move along a preset direction (e.g., an axial direction of the scanning cavity 323-1).

In some embodiments, as shown in FIG. 3, the imaging system 300 may further include a connector 324-1 that may be mounted on the imaging module 320. In some embodiments, the transfer module 330 may transfer the scanning cabin 340 from the scanning cabin management module 310 to the imaging module 320, and the scanning cabin 340 may be mounted on the scanning movement unit 324 of the imaging module 320 through the connector 324-1. Specifically, the connector 324-1 may be detachably mounted on the scanning movement unit 324, and the scanning movement unit 324 may drive the scanning cabin 340 along a preset direction through the connector 324-1. Further, in some embodiments, the scanning cabin 340 may be electrically connected to the imaging module 320 through the g connector 324-1 to enable the imaging module 320 to obtain, through the connector 324-1, at least one of the anesthesia parameter, the temperature parameter, the injection parameter, and the physiological parameter information of the subject within the scanning cabin 340. In some embodiments, the connector 324-1 may have a pipeline (not shown in FIG. 3) and a circuit (not shown in FIG. 3). In some embodiments, the scanning cabin 340 may be electrically connected to the imaging module 320 through the circuit of the connector 324-1, and at least one of the anesthesia parameter, the temperature parameter, the injection parameter, and the physiological parameter information (a respiratory signal, a blood oxygenation signal, and a temperature signal, etc.) of the subject may be transmitted through the circuit to the imaging module 320. In some embodiments, the scanning cabin 340 may be connected to the imaging module 320 through a pipeline of the connector 324-1, and the imaging module 320 may pass an anesthesia gas, a temperature-controlled medium, or an injected medication into the scanning cabin 340 through the pipeline of the connector 324-1.

In some embodiments, as shown in FIG. 1, the scanning cabin management module 310 may include a mounting base 312 and a rotating substrate 313, the rotating substrate 313 may be rotatably mounted to the mounting base 312, and the cabin seats 311 may be provided on the rotating substrate 313 along the circumference direction of the rotating substrate 313. In some embodiments, when one of the scanning cabins 340 needs to be transferred to the imaging module 320 by the transfer module 330, the scanning cabin 340 may be rotatably moved to the side of the scanning cabin management module 310 near the transfer module 330 through the rotating substrate 313, and then the scanning cabin 340 may be moved through the transfer module 330. With the above setting, the convenience of the transfer module 330 moving the scanning cabin 340 may be greatly improved.

Further, in some embodiments, as shown in FIG. 1, an assembly cavity 312-1 may be provided within the mounting base 312, and the rotating substrate 313 may be mounted on a sidewall of the assembly cavity 312-1. In some embodiments, the rotating substrate 313 may be provided with a plurality of through holes, the plurality of through holes may form the plurality of cabin seats 311. Specifically, each of the plurality of through holes may form one of the plurality of cabin seats 311. In some embodiments, the scanning cabins 340 may be placed into each of the plurality of cabin seats 311 through an outer side of the rotating substrate 313, and fully into the assembly cavity 321. In some embodiments, one side of the mounting base 312 proximate to the transfer module 330 may be provided with a sampling port 312-2, and the transfer module 330 may be configured to clamp the scanning cabin 340 within the assembly cavity 312-1 through the sampling port 312-2.

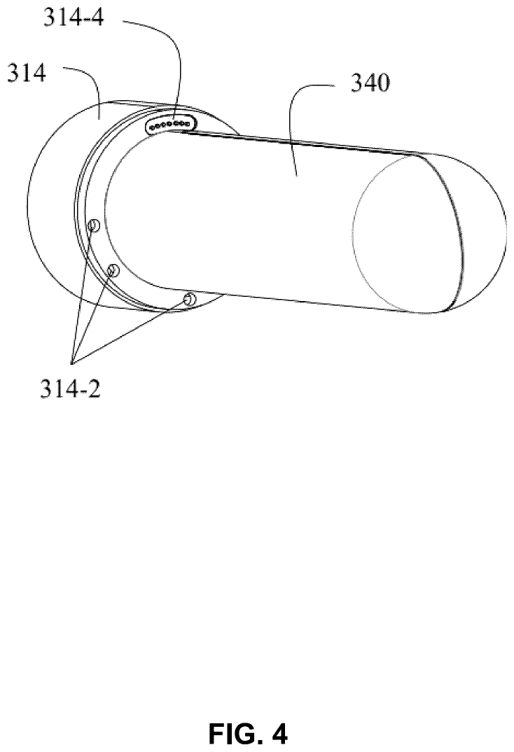

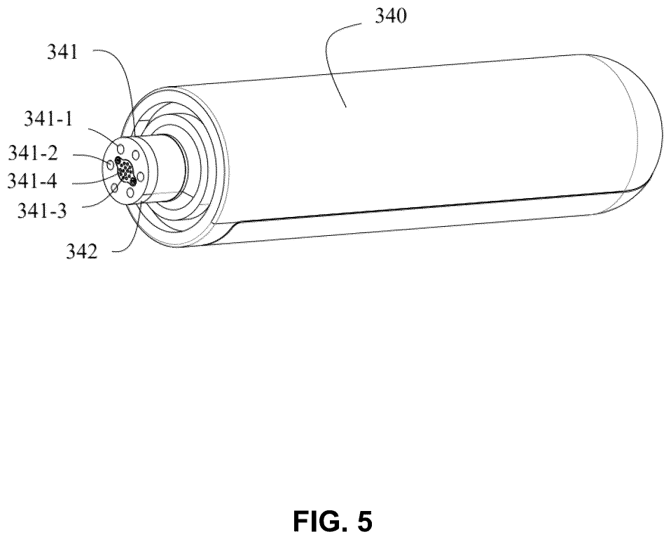

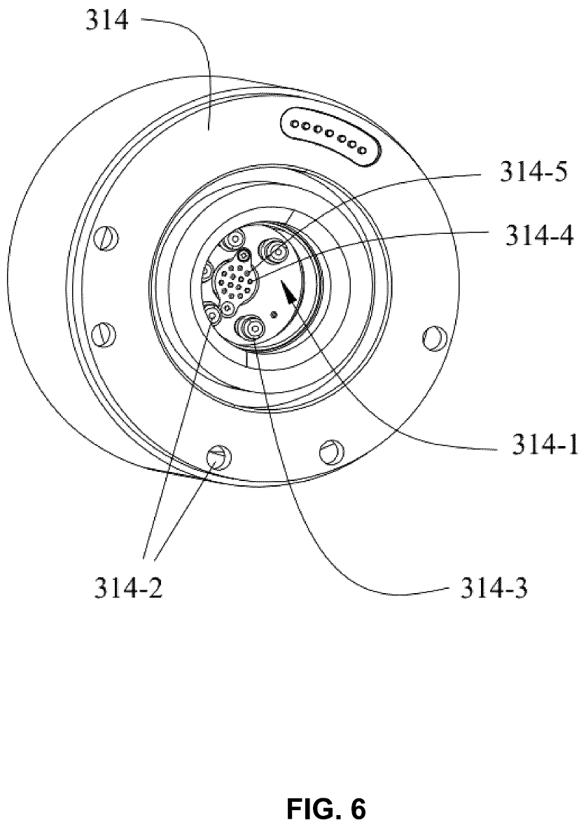

In order to improve an assembly efficiency of the scanning cabin 340 with the rotating substrate 313, in some embodiments, the scanning cabin management module 310 may further include an adapter 314, and the scanning cabin 340 may be rotatably mounted on the rotating substrate 313 relative to the rotating substrate 313 through the adapter 314. For example, FIG. 4 is a schematic diagram illustrating the installation of the adapter 314 and a scanning cabin 340 according to some embodiments of the present disclosure. FIG. 5 is a schematic diagram illustrating f an exemplary scanning cabin 340 according to some embodiments of the present disclosure. FIG. 6 is a schematic diagram illustrating an exemplary adapter 314 according to some embodiments of the present disclosure. The structure of the adapter and the scanning cabin involved in the present disclosure embodiments is described in detail below in connection with FIG. 4-FIG. 6. It should be noted that the following embodiments are merely provided for the purpose of explaining the present disclosure and do not constitute a limitation of the present disclosure.

Specifically, as shown in FIG. 5, the scanning cabin 340 may be provided with a docking connector 341, and as shown in FIG. 6, the adapter 314 may be provided with a docking hole 314-1 adapted to the docking connector 341. In some embodiments, the docking hole 314-1 adapted to the docking connector 341 may be that a diameter of the docking hole 314-1 is larger than a diameter of the docking connector 341 and a difference between the diameter of the docking hole 314-1 and the diameter of the docking connector 341 is within a first preset threshold range (e.g., 1 mm, 3 mm, or 5 mm) such that the docking connector 341 may be fixedly mounted to the docking hole 314-1. In some embodiments, the docking connector 341 may be threaded or snap-fit connected to the docking hole 314-1 for fixed installation.

In some embodiments, as shown in FIG. 4-FIG. 6, the docking connector 341 may be provided with a first fluid transfer channel 341-1 in flow communication with an interior of the scanning cabin 340, and the adapter 314 may be provided with a second fluid transfer channel 314-2 in flow communication with the first fluid transfer channel 341-1. The external temperature-control medium, the injection medicine, and the anesthesia gas may sequentially pass through the second fluid transfer channel 314-2 and the first fluid transmission channel 341-1 into the interior of the scanning cabin 340.

In some embodiments, as shown in FIG. 4-FIG. 6, the docking connector 341 may be provided with a communication recess 341-2 in flow communication with the first fluid transfer channel 341-1 on an end face of a side of the docking connector 341 proximate to the bottom wall of the docking hole 314-1, and the communication recess 341-2 may be provided with one or more holes at the bottom portion of the communication recess 341-2, through which the communication recess 341-2 is in flow communication with the first fluid transfer channel 341-1. Correspondingly, the bottom wall of the docking hole 314-1 may be provided with a communication protrusion 314-3 adapted to the communication recess 341-2, and an end opening of the second fluid transfer channel 314-2 may be provided on the communication protrusion 314-3, so that the second fluid transfer channel 314-2 may be in flow communication with the first fluid transfer channel 341-1 through the communication protrusion 314-3 and the communication recess 341-2 when the communication protrusion 314-3 is inserted inside the communication recess 341-2. In some embodiments, the communication protrusion 314-3 adapted to the communication recess 341-2 may be such that the diameter of the communication protrusion 314-3 is less than the diameter of the communication recess 341-2, and a difference between the diameter of the communication recess 341-2 and the diameter of the communication protrusion 314-3 is within a second preset threshold range (e.g., 1 mm, 3 mm, or 5 mm) such that the communication protrusion 314-3 may be inserted to the communication recess 341-2. Further, in some embodiments, the docking connector 41 may be provided with a plurality of communication recesses 341-2 on an end face of a side of the docking connector 41 proximate the bottom wall of the docking hole 314-1, and accordingly, the bottom wall of the docking hole 314-1 may be provided with a plurality of communication protrusions 314-3 adapted to the plurality of communication recesses 341-2, and it should be appreciated that the plurality of first fluid transfer channels 341-1 and the plurality of second fluid transfer channels 314-2 may form an anesthesia gas line, a medicine injection line, a temperature control gas line, and the like.

In some embodiments, the interior of the scanning cabin 340 may be provided with a sensing assembly (not shown in the figure) configured to obtain information of a plurality of physiological parameters (also referred to as physiological parameter information) such as the respiratory signal, the blood oxygen signal, and the body temperature signal, an anesthesia parameter, a temperature parameter, and an injection parameter of the subject. In some embodiments, as shown in FIG. 5-FIG. 6, the docking connector 341 may be provided with a first electrical connection line 341-3 electrically connecting the sensing assembly inside the scanning cabin 340, and the adapter 314 may be provided with a second electrical connection line 314-4 capable of conducting the first electrical connection line 341-3. In some embodiments, at least one of the physiological parameter information, the anesthesia parameter, the temperature parameter, and the injection parameter of the subject may be transmitted outwardly through the first electrical connection line 341-3 and the second electrical connection line 314-4 in sequence.

In some embodiments, as shown in FIG. 4-FIG. 6, the docking connector 341 may be provided with a conduction recess 341-4 on an end face near the bottom wall of the docking connector 314-1 that conducts the first electrical connection line 341-3, and one end of the first electrical connection line 341-3 may be electrically connected to the conduction recess 341-4. Correspondingly, the bottom wall of the docking hole 314-1 may be provided with a conduction protrusion 314-5 adapted to the conduction recess 341-4, and one end of the second electrical connection line 314-4 may be electrically connected to the conduction protrusion 314-5, so that when the conduction protrusion 314-5 is inserted into the conduction recess 341-4, the second electrical connection line 314-4 is capable of being electrically connected to the first electrical connection line 341-3 through the conduction protrusion 314-5 and the conduction recess 341-4. In some embodiments, the conduction protrusion 314-5 adapted to the conduction recess 341-4 may be that the diameter of the conduction protrusion 314-5 is smaller than the diameter of the conduction recess 341-4, and a difference between the diameter of the conduction protrusion 314-5 and the diameter of the conduction recess 341-4 is within a third preset threshold range (e.g., 1 mm, 3 mm, or 5 mm), such that the conduction protrusion 314-5 may be inserted into the conduction recess 341-4. Further, in some embodiments, an end face of a side of the docking connector 341 proximate to the bottom wall of the docking hole 314-1 may be provided with a plurality of conduction recesses 341-4, and accordingly, the bottom wall of the docking hole 314-1 may be provided with a plurality of conduction protrusions 314-5 adapted to the plurality of conduction recesses 341-4. It may be appreciated that through a corresponding fit of the plurality of conduction recesses 341-4 and the plurality of conduction protrusions 314-5, a plurality of physiological parameter signals such as the respiratory signals, the blood oxygen signals, and the body temperature signals, and the anesthesia parameters, the temperature parameters, and the injection parameters may be transmitted simultaneously.

For ease of understanding, the assembly process of the scanning cabin 340 is described below in connection with FIG. 3-FIG. 6. As shown in FIG. 4-FIG. 6, the subject is loaded into the scanning cabin 340. Then, the scanning cabin 340 in which the subject is placed is mounted on the adapter 314 through the mounting fit of the adapter 341 and the docking hole 314-1 as shown in FIG. 4. In some embodiments, in order to facilitate a staff to pick up the scanning cabin 340 assembled with the adapter 314, as shown in FIG. 3, a handle 360 is fixedly provided on a side of the adapter 314 back from the scanning cabin 340. Afterwards, the staff may push the scanning cabin 340 assembled with the adapter 314 from a side of the scanning cabin management module 310 into the cabin seat 311 by the handle 360. And, as shown in FIG. 3, the transfer module 330 may clamp the scanning cabin 340 within the assembly cavity 312-1 through the sampling port 312-2 and place the scanning cabin 340 on the connector 324-1 of the scanning movement unit 324.

Further, to facilitate the connection of the scanning cabin 340 and the adapter 314, in some embodiments, as shown in FIG. 5, the scanning cabin 340 may be removably connected to the adapter 314 through a locking structure 342 (e.g., a motorized locking structure, etc.). In some embodiments, the locking structure 342 may be a motorized magnetic suction structure, a motorized snap-fit structure, etc.

In some embodiments, the imaging system 300 may further include a function assistance module (not shown in FIG. 3). In some embodiments, the function assistance module may include at least one of the anesthesia device, the temperature control device, the injection device, or the physiological monitoring device (not shown in the figure). In some embodiments, a portion of the function assistance module may be provided at least within the scanning cabin 340 and another portion of the function assistance module may be provided on the scanning cabin management module 310 or the imaging module 320.

In some embodiments, the physiological monitoring device may be disposed within the scanning cabin 340, and the physiological monitoring device may be configured to monitor the physiological parameter of the subject that is placed within the scanning cabin 340. In some embodiments, the physiological monitoring device may be configured to monitor physiological parameter information such as a heartbeat signal, a respiratory signal, a blood oxygen signal, a body temperature signal, etc., of the subject. Specifically, the physiological monitoring device may include a plurality of sensing assemblies (not shown in the figure) and a signal processing assembly (not shown in the figure) electrically connected to different sensing assemblies respectively. The plurality of sensing assemblies may be configured to obtain the physiological parameter information such as the respiratory signal, the blood oxygen signal, and the body temperature signal of the subject. The plurality of sensing assemblies may be configured to transmit the plurality of obtained physiological parameter information to the signal processing assembly. In some embodiments, when the scanning cabin is located on the scanning cabin management module 310, the signal processing assembly may transmit the physiological parameter information to the scanning cabin management module 310 or the control module through the adapter 314 and the docking connector 341. In some embodiments, when the scanning cabin is located on the imaging module 320, the signal processing assembly may transmit the physiological parameter information to the imaging module 320 or the control module through the connector 324-1 and the docking connector 341.

In some embodiments, the temperature control device may be disposed within the scanning cabin 340, and the temperature control device may be configured to detect an actual temperature within the scanning cabin 340 and adjust the actual temperature of the scanning cabin 340. In some embodiments, the temperature control device may include a heating pad or a heating pipeline to heat the scanning cabin 340. In some embodiments, the temperature control device may include a temperature detection assembly (not shown in the figure) and a signal processing assembly (not shown in the figure) electrically connected to the temperature detection assembly. The temperature detection assembly may be configured to obtain the actual temperature of the scanning cabin 340, and the temperature detection assembly may be capable of transmitting the obtained temperature signal to the signal processing assembly. In some embodiments, when the scanning cabin is located on the scanning cabin management module 310, the signal processing assembly may transmit the temperature signal to the scanning cabin management module 310 or the control module through the adapter 314 and the docking connector 341. In some embodiments, when the scanning cabin is located on the imaging module 320, the signal processing assembly may transmit the temperature signal to the imaging module 320 or the control module through the connector 324-1 and the docking connector 341.

In some embodiments, a portion of the injection device may be disposed within the scanning cabin 340, and another portion of the injection device may be disposed on the scanning cabin management module 310 or the imaging module 320. The injection device may be configured to perform the injection on the subject within the scanning cabin 340 and to detect an actual injection dosage or an actual injection speed. In some embodiments, the injection device may include an injection dosage detection assembly (not shown in the figure) and a signal processing assembly (not shown in the figure) electrically connected to the injection dosage detection assembly. The injection dosage detection assembly may be configured to obtain an injection dosage, and the injection dosage detection assembly being capable of transmitting an obtained injection dosage signal to the signal processing assembly. In some embodiments, the injection device may include an injection speed detection assembly (not shown in the figure) and the signal processing assembly (not shown in the figure) electrically connected to the injection speed detection assembly, the injection speed detection assembly may be configured to obtain an actual injection speed and transmit an obtained injection speed signal to the signal processing assembly. In some embodiments, when the scanning cabin is located on the scanning cabin management module 310, the signal processing assembly may transmit the injection dosage signal or the injection speed signal to the scanning cabin management module 310 or the control module through the adapter 314 and the docking connector 341. In some embodiments, when the scanning cabin is located on the imaging module 320, the signal processing assembly may transmit the injection dosage signal or the injection speed signal to the imaging module 320 or the control module through the connector 324-1 and the docking connector 341. In some embodiments, the injection dosage detection assembly and/or the injection speed detection assembly may be provided within the scanning cabin 340. In some embodiments, the injection device may further include an injection medicine canister, the injection medicine canister may be provided on the scanning cabin management module 310 or the imaging module 320 and be in flow communication with the scanning cabin 340 through the adapter 314 or the connector 324-1.

In some embodiments, a portion of the anesthesia device may be configured to be disposed within the scanning cabin 340, and another portion of the anesthesia device is disposed on the scanning cabin management module 310 or the imaging module 320, and the anesthesia device may be configured to output the anesthesia gas to the scanning cabin 340 and detect the actual concentration of the anesthesia gas within the scanning cabin 340. In some embodiments, the anesthesia device may include an anesthesia detection assembly (not shown in the figure) and a signal processing assembly (not shown in the figure) electrically connected to the anesthesia detection assembly, the anesthesia detection assembly may be configured to obtain an actual concentration of the anesthesia gas within the scanning cabin 340 and transmit the obtained anesthesia signal to the signal processing assembly. In some embodiments, when the scanning cabin is located on the scanning cabin management module 310, the signal processing assembly may transmit the anesthesia signal to the scanning cabin management module 310 or the control module through the adapter 314 and the docking connector 341. In some embodiments, when the scanning cabin is located on the imaging module 320, the signal processing assembly may transmit the anesthesia signal to the imaging module 320 or the control module through the connector 324-1 and the docking connector 341. In some embodiments, the anesthesia detection assembly may be disposed within the scanning cabin 340. In some embodiments, the anesthesia device may further include a volatile tank in which anesthesia gas is stored, and the volatile tank may be provided on the scanning cabin management module 310 or the imaging module 320 and be in flow communication with the scanning cabin 340 through the adapter 314 or the connector 324-1.

Referring back to FIG. 3, in order to more clearly display the plurality of physiological parameters, the anesthesia parameter, the temperature parameter, and the injection parameter of the subject, in some embodiments, as shown in FIG. 3, the scanning cabin management module 310 may further include a display screen 315 configured to display at least one of the plurality of physiological parameters, the anesthesia parameter, the temperature parameter, and the injection parameter of the subject. The display screen 315 may be disposed in the center region of the mounting base 312. In some embodiments, the rotating substrate 313 may be in a circular shape, the display screen 315 may be disposed in the center region of the rotating substrate 313, and the plurality of cabin seats 311 may be distributed on the rotating substrate 313 along a circumferential direction surrounding the display screen 315. Further, in some embodiments, the display screen 315 may be electrically connected to an electrical signal processing assembly, and the electrical signal processing assembly may be capable of converting an obtained electrical signal into a graphic signal to be displayed on the display screen 315. By providing the display screen 315, it may be greatly facilitated for the staff to intuitively obtain a real-time state of the subject and environmental data (e.g., temperature information, concentration of the anesthesia gas, etc.) within the scanning cabin 340 through the display screen 315. In some embodiments, the scanning cabin management module 310 may have an interaction device (not shown in the figure), and the interaction device has an interaction function. The staff may control and manage the scanning cabin 340 through the interaction device on the scanning cabin management module 310. In some embodiments, the interaction device may include a virtual button provided on the display screen 315, a physical button, a voice interaction device, etc., or a combination thereof. In some embodiments, the interaction device may be integrated into the control module.

FIG. 7 is a schematic diagram illustrating an exemplary transfer module 350 according to some embodiments of the present disclosure.

In some embodiments, as shown in FIG. 7, the transfer module 330 may include a displacement unit 331 and a mechanical arm unit 332. The displacement unit 331 may be able to drive the mechanical arm unit 332 to perform a translation. The mechanical arm unit 332 may be connected to the displacement unit 331 at one end, and the other end of the mechanical arm unit may be configured to clamp the scanning cabin 340. Through the cooperation of the displacement unit 331 and the mechanical arm unit 332, the scanning cabin 340 may be clamped and moved, effectively enhancing the transfer efficiency of the transfer module 330. Further, in some embodiments, the displacement unit 331 may be capable of driving the mechanical arm unit 332 to translate along a preset direction. In some embodiments, the preset direction may be the length direction of the scanning cabin 340, i.e., the preset direction is an axial direction of the scanning cavity 323-1. With the above setting, the displacement unit 331 may drive the scanning cabin 340 to be inserted into the connector 324-1 along the axial direction of the scanning cavity 323-1 through the mechanical arm unit 332.

In some embodiments, as shown in FIG. 7, the mechanical arm unit 332 may include a clamping assembly 332-1 and a rotating arm assembly 332-2. One end of the rotating arm assembly 332-2 may be connected to the clamping assembly 332-1, and the other end of the displacement unit 331 may be rotatably connected to the displacement unit 331. With the above setup, the rotating arm assembly 332-2 may be able to drive the clamping assembly 332-1 to move to a preset position, so that the clamping assembly 332-1 is convenient to clamp the scanning cabin 340.

In some embodiments, as shown in FIG. 7, the rotating arm assembly 332-2 may include a connecting arm 332-21 and a first rotating motor 332-22. The first rotating motor 332-22 may be mounted to the displacement unit 331, the connecting arm 332-21 may connect the first rotating motor 332-22 to the clamping assembly 332-1, and the first rotating motor 332-22 may be able to drive the clamping assembly 332-1 to rotate through the connecting arm 332-21. Through the above setting, the first rotating motor 332-22 may drive the connecting arm 332-21 to rotate in a wide range along an output axis of the first rotating motor 332-22, so that the clamping assembly 332-1 rotates along the output axis of the first rotating motor 332-22 in the wide range.

In some embodiments, as shown in FIG. 7, the clamping assembly 332-1 may include a driving component 332-11 and a pair of clamping jaws 332-13 oppositely disposed (also referred to as the jaws 332-13). The driving component 332-11 may connect the pair of clamping jaws 332-13 oppositely disposed and the rotating arm assembly 332-2. The driving component 332-11 may be capable of driving the pair of clamping jaws 332-13 oppositely disposed to rotate relative to the rotating arm assembly 332-2, and the driving component 332-11 may drive the pair of clamping jaws 332-13 to clamp or open. Specifically, in some embodiments, the driving component 332-11 may include a driving motor (not shown in FIG. 7), a worm rod (not shown in FIG. 7), and a pair of worm gears (not shown in FIG. 7). The worm gear may be connected to the output shaft of the driving motor, and each of a pair of worm gears oppositely disposed may be meshed and connected to each of both sides of the worm rod, respectively, and the worm gears disposed on the both sides of the worm rod may be respectively fixedly connected to the pair of clamping jaws 332-13. In some embodiments, as shown in FIG. 7, the driving component 332-11 may further include a second rotating motor 332-12, the second rotating motor 332-12 may be capable of driving the clamping jaws 332-13, the driving motor, the worm rod, and the worm gears to rotate synchronously relative to the rotating arm assembly 332-2. In some embodiments, the clamping jaws 332-13 may also rely on their own elastic deformation to realize the open and close the clamping jaws 332-13.

In some embodiments, as shown in FIG. 7, the displacement unit 331 may include a linear actuator 331-1 and a mounting slider 331-2. One end of the mechanical arm unit 332 may be mounted to the mounting slider 331-2, and the linear actuator 331-1 may be able to drive the mounting slider 331-2 to drive the mechanical arm unit 332 to move along the length direction of the scanning cabin 340.

The embodiments of the present disclosure also provide an imaging process, including: mounting each of the plurality of scanning cabins 340 in which a subject is placed to one of the plurality of corresponding cabin seats 311 of the scanning cabin management module 310, monitoring a physiological parameter of the subject that is placed in the scanning cabin 340 using a physiological monitoring device, or outputting the anesthesia gas to the scanning cabin 340 using the anesthesia device, or detecting an actual concentration of the anesthesia gas in the scanning cabin 340 using the anesthesia detection device, or adjusting the temperature of the scanning cabin 340 using the temperature control device and detecting the actual temperature of the scanning cabin 340 using the temperature detection assembly, or injecting the medicine to the subject within the scanning cabin using the injection device and detecting an actual injection dosage using the injection dosage detection assembly, and/or detecting an actual injection speed within the scanning cabin 340 using the injection speed detection assembly, transferring the at least one scanning cabin 340 from the scanning cabin management module 310 to the imaging module 320 using the transfer module 330, and performing the scanning on the subject within the scanning cabin 340 transferred to the imaging module 320 using the imaging module 320.

In some embodiments, the imaging system may further include a control module (not shown in FIG. 3-FIG. 7) electrically connected to the scanning cabin management module 310, the imaging module 320, and the transfer module 330, respectively, and the control module may be capable of controlling the operation of the scanning cabin management module 310, the imaging module 320, and the transfer module 330, respectively. It should be noted that the scanning cabin management module 310, the imaging module 320, and the transfer module 330 may be provided with a controller in correspondence, respectively, and the scanning cabin management module 310, the imaging module 320, and the transfer module 330 may also all be provided with a controller in correspondence with only one controller.

In some embodiments, when performing an imaging module, or “animal imaging device” or “animal imaging system” (e.g., CT, SPECT, PET, MR, and any combination of modalities) on the animal, the animal needs to be anesthetized. It is necessary to anesthetize the animal to avoid a poor imaging result and motion artifact caused by a physical movement of the animal during the scanning. After the animal is in the anesthesia state, the body temperature of the animal may gradually decrease, and a prolonged hypothermia may lead to the death of the animal. Therefore, the animal imaging device needs to be configured with the anesthesia device and the heat preservation device together to maintain the animal in a suitable life state.

Currently, the existing animal imaging device is not linked with the anesthesia device and the heat preservation device, and the experimenter or staff need to manually set the concentration of the anesthesia gas and the heat preservation temperature (or the “temperature within the scanning cabin”) in advance before performing the scanning on the animal, which is a complicated operation procedure. Both the concentration of the anesthesia gas and the heat preservation temperature may affect the heartbeat rate of the animal, thereby affecting the metabolic state of the animal. For SPECT and PET imaging devices, the metabolic state of the animal may affect the imaging result, so it is necessary to try to ensure that the concentration of the anesthesia gas and the heat preservation temperature are the same in the repeated experiments.

When scanning an animal using a CT or MR imaging device, for some applications, it is necessary to inject the animal with a contrast agent to improve the contrast in specific parts of the image. When scanning an animal using a SPECT or PET imaging device, it is necessary to inject the animal with a radionuclide medicine. The animal, after being injected with the contrast agent, may have an allergic reaction, which, in response to determining that left untreated, may affect the scanning result. Therefore, the animal imaging device needs to be equipped with an injection device used in conjunction with the physiological monitoring device and the temperature control device to maintain the animal in a suitable vital state. After injecting the contrast agent or radionuclide medicine into the animal, the experimenter is generally required to manually fill in the injection information (e.g., medicine information, injection time and injection dose) into the experimental registration information (i.e., including scanning-related information), and it is necessary to artificially control the start of the scanning process after the injection of the medicine, which makes the experimental process complicated to operate with a low degree of automation.

Therefore, based on the above prior art, it becomes particularly important to link the animal imaging device with the anesthesia device and the temperature control device, and link at least two of the injection device, the anesthesia device, the temperature control device, the physiological monitoring device, the scanning cabin management module, the transfer module, and the imaging module, to realize the full automation of the management, anesthesia, heat preservation, injection, transfer, and scanning of the animal, which avoids an increase of experimental errors caused by human manual operation, and also simplifies the whole workflow and has a great development prospect.

FIG. 8 is a flowchart illustrating an exemplary imaging system 800 according to some embodiments of the present disclosure.

Referring to FIG. 8, in some embodiments, the imaging system 800 may include a control module 810, an imaging module 820, anesthesia device 830, and a temperature control device 840, and the control module 810 may be electrically connected to the imaging module 820, the anesthesia device 830, and the temperature control device 840, respectively. In some embodiments, the imaging system 800 may further include a scanning cabin management module (not shown in FIG. 8). In some embodiments, the anesthesia device 830 may be configured to receive a first control signal sent by the control module 810, output the anesthesia gas to the scanning cabin (or referred to as a “scanning cabin”) based on the first control signal, and feed the anesthesia signal back to the control module 810. In some embodiments, the temperature control device 840 may be configured to receive the second control signal sent by the control module 810, adjust the temperature of an scanning cabin based on the second control signal, and feed a heat preservation signal or a temperature signal to the control module 810 characterizing the heat preservation condition of the animal, e.g., the actual temperature in the scanning cabin, the actual body temperature of the animal, or the actual temperature of the heating device, etc., detected by the temperature detection assembly in the temperature control device 840. In some embodiments, the imaging module 820 may be configured to receive a sixth control signal sent by the control module 810, and perform the imaging on the animal in the scanning cabin based on the sixth control signal. In some embodiments, the control module 810 may receive the anesthesia signal and the temperature control signal, and send a corresponding control signal to control the anesthesia device 830, the temperature control device 840, the imaging module 820, and the scanning cabin management module to perform a corresponding action.

In some embodiments, the control module 810 may be electrically connected with the imaging module 820 and the scanning cabin management module through a cable. The imaging module 820 and the scanning cabin management module are placed in a scanning room (i.e., a venue where the scanning operation is performed), and the control module 810 may be placed outside of the scanning room to maintain control with the imaging module 820 and the scanning cabin management module through electrical signals. In some embodiments, the control module 810 may be disposed in an operation room (i.e., a venue where the operator controls the scanning operation). In some embodiments, the operation room may also include a server, an operating device, a display device (not shown in FIG. 8), and the like. In some embodiments, an operator may use an operating device in the operator room to perform a corresponding manual control on the anesthesia device 830, the temperature control device 840, the imaging module 820, and the scanning cabin management module. In some embodiments, the control module 810 may also be provided in the scanning room, integrated on the imaging module 820 or the scanning cabin management module. In some embodiments, the control module 810 may also include a mobile device to perform a remote control on the imaging module 820 and the scanning cabin management module.

By linking the control module 810 with the imaging module 820, the scanning cabin management module, the anesthesia device 830, and the temperature control device 840, the anesthesia, heat preservation, and scanning of the animal are thus made to be automatically controlled, which reduces the complexity of the entire a scanning operation process, improves the reproducibility and precision of the experiments, and is more friendly to the operator.

In some embodiments, the control module 810 being electrically connected to the imaging module 820, the anesthesia device 830, and the temperature control device 840 respectively, means that the control module 810 is connected to the imaging module 820, the scanning cabin management module (e.g., the scanning cabin management module 210 as shown in FIG. 2), the anesthesia device 830, and the temperature control device 840 with a cable, respectively, and once the cable is energized, the control module 810 is electrically connected to the imaging module 820, the scanning cabin management module, the anesthesia device 830, and the temperature control device, respectively, thereby realizing am electrical connection. When performing the animal scanning, after performing a preliminary preparation, an operator may select a scanning protocol on the operating device, and after the operator manually or the control module 810 automatically starts the system, the control module 810 may send a preset setting value in the scanning protocol to the anesthesia device 830 and the temperature control device 840, respectively, and begin to pass the anesthesia gas into the scanning cabin and keep the scanning cabin warm to ensure that the body temperature of the animal may not be decrease. After the scanning cabin is transferred to the imaging module 820, the control module 810 may start the imaging module 820 to perform a scanning according to a preset scanning start time in the scanning protocol. In some embodiments, in response to determining that an injection is to be performed on the animal, the control module 810 may send a preset injection setting value in the scanning protocol to the injection device to start injecting the animal, and then start the imaging module 820 to perform the scanning after the injection is completed. In some embodiments, when performing an animal scanning t, after performing the preliminary preparation, the operator may also remotely select the scanning protocol and start the system through the mobile device, so that the imaging system 800 may automatically perform the operations of anesthesia, holding, injection, and scanning.

In some embodiments, the imaging module 820 may include an imaging device, e.g., CT, SPECT, PET, MR, or a combination of each of these modalities. More descriptions of the imaging module 820 may be found in the descriptions of other portions of the present disclosure and will not be repeated herein.

In some embodiments, when the scanning cabin is on the scanning cabin management module, the animal may be anesthetized. In some embodiments, the animal may be anesthetized based on an animal physiological parameter when the scanning cabin is located on the imaging module. In some embodiments, the anesthesia device 830 may include a volatile tank connected to the scanning cabin through the pipeline. In some embodiments, an anesthesia inlet and an anesthesia outlet connected to an inner cavity (not shown in FIG. 8) of the scanning cabin may be opened at one end of the scanning cabin, the volatile tank may be connected to the anesthesia inlet and the anesthesia outlet respectively through the pipeline (not shown in FIG. 8), the anesthesia gas is output from the volatile tank, the anesthesia gas is transported to the anesthesia inlet through the pipeline, the anesthesia gas enters into the inner cavity of the scanning cabin through the anesthesia inlet to perform a continuous inhalation anesthesia on the animal in the inner cavity, and the residual anesthesia gas after the scanning is discharged back to the volatile tank, to avoid the effect of the leakage of the anesthesia gas. More descriptions of the anesthesia inlet and the anesthesia outlet may be found in the description of the first fluid transfer channel 341-1 in FIG. 5, and will not be repeated herein.

In some embodiments, to automate control of the overall experimental operation process, the concentration or dosage of the anesthesia gas in the inner cavity of the scanning cabin may be monitored and electrically adjustable. In some embodiments, the first control signal may include an anesthesia gas parameter. In some embodiments, the anesthesia gas parameter may include at least one of a concentration parameter of the anesthesia gas, a dosage parameter of the anesthesia gas, or an output rate parameter of the anesthesia gas. In some embodiments, the anesthesia device 830 may output the anesthesia gas to the scanning cabin based on the anesthesia gas parameter.

In some embodiments, the anesthesia device 830 may include an anesthesia detection assembly configured to detect the actual concentration of the anesthesia gas or the actual dosage of the anesthesia gas in the anesthesia device 830. Since the concentration of the anesthesia gas or the dosage of the anesthesia gas required for different types of animals, different body sizes of animals, different weights of animals, different physiological states of animals, and different types of anesthetics are different, the concentration of the anesthesia gas or the dosage of the anesthesia gas needs to be strictly controlled. The concentration of the anesthesia gas or the dosage of the anesthesia gas that is too low may cause the under-anesthesia animal to wake up during the experiment, which may affect the imaging effect. The concentration of the anesthesia gas or the dosage of the anesthesia gas that is too high may even jeopardize the life of the animal. Therefore, according to the different conditions of the animal, it is necessary to input different anesthesia thresholds in the anesthesia device 830. An anesthesia threshold is a range value of the suitable concentration of the anesthesia gas or a range value of the suitable dosage of the anesthesia gas. In some embodiments, the anesthesia thresholds may be set by an operator in advance based on experience and a safety standard based on information about different species and different states of the animals. In some embodiments, the anesthesia thresholds corresponding to animals of different species and different states may also be obtained based on big data analysis. In some embodiments, the anesthesia thresholds and the corresponding animal state information may be made in advance as a scanning protocol stored in the imaging system, and the corresponding anesthesia thresholds in the scanning protocols may be automatically recognized at the start of the scanning based on the animal state information. In some embodiments, the scanning protocol may include a type of the anesthetic agent, the anesthesia threshold, species of the animal, a weight of the animal, an average body temperature of the animal, an average heartbeat rate of the animal, and the like.

In some embodiments, the anesthesia device 830 may compare the actual concentration of the anesthesia gas or the actual dosage of the anesthesia gas in the anesthesia device 830 detected by the anesthesia detection assembly with the anesthesia threshold. In some embodiments, the anesthesia detection assembly may feed a detection signal to the anesthesia device 830 when the actual concentration of the anesthesia gas or the actual dosage of the anesthesia gas exceeds the anesthesia threshold, that is, when the actual concentration of the anesthesia gas exceeds a range value of a suitable concentration of the anesthesia gas, the anesthesia detection assembly may feed the detection signal back to the anesthesia device 830. In some embodiments, the anesthesia device 830 may adjust the output rate of the anesthesia gas to adjust the actual concentration of the anesthesia gas to be within the anesthesia threshold. By adjusting the actual concentration of the anesthesia gas to be within the anesthesia threshold, the concentration of the anesthesia gas in the scanning cabin may be electrically controlled and adjustable, avoiding an over-anesthesia or under-anesthesia of the animal caused by the actual concentration of the anesthesia gas exceeding the anesthesia threshold, which may not only protect the life safety of the animal, but also obtain a better imaging effect.

In some embodiments, the anesthesia detection assembly may be provided in the scanning cabin to detect the concentration of the anesthesia gas or the dosage of the anesthesia gas in the scanning cabin, and then compare the actual concentration of the anesthesia gas or the actual dosage of the anesthesia gas in the scanning cabin detected by the anesthesia detection assembly with the anesthesia threshold. In some embodiments, the anesthesia detection assembly may feed the detection signal back to the anesthesia device 830 when the actual concentration of the anesthesia gas or the actual dosage of the anesthesia gas exceeds the anesthesia threshold, i.e., when the actual concentration of the anesthesia gas exceeds a suitable concentration of the anesthesia gas or when the actual dosage of the anesthesia gas exceeds a suitable dosage of the anesthesia gas. In some embodiments, the anesthesia device 830 may adjust the actual concentration of the anesthesia gas or the actual dosage of the anesthesia gas to be within the anesthesia threshold by adjusting the flow rate for outputting the anesthesia gas.

After anesthetizing the animal, the body temperature of the animal may gradually decrease when the animal is in the anesthesia state, and the prolonged hypothermia may lead to the death of the animal. Therefore, the anesthesia device 830 needs to be used in conjunction with the temperature control device 840 to maintain the animal in a suitable life state.

In some embodiments, after the anesthesia device 830 completes anesthetizing the animals in the scanning cabin, the anesthesia device 830 may feed the anesthesia signal back to the control module 810, and the anesthesia signal may characterize the anesthesia condition of the scanning cabin. In some embodiments, the control module 810 may receive the anesthesia signal fed back by the anesthesia device 830 after completing the anesthesia, and then send the second control signal to the temperature control device 840, and the temperature control device 840 may keep the animal warm based on the second control signal. In some embodiments, after receiving the anesthesia signal fed back by the anesthesia device 830 after completing the anesthesia, the control module 810 may send the anesthesia signal to a terminal device (e.g., the one or more terminals 130 or the display screen 315) to remotely control the temperature control device 840 by an operator to keep the animal warm.

In some embodiments, when the scanning cabin is located on the scanning cabin management module, the anesthesia detection assembly may transmit the anesthesia signal to the scanning cabin management module or the control module 810 through an adapter and a docking connector (not shown in FIG. 8). In some embodiments, when the scanning cabin is located on the imaging module, the anesthesia detection assembly may transmit the anesthesia signal to the imaging module or the control module 810 through the connector and the docking connector. In some embodiments, after receiving the anesthesia signal, the control module 810 may send a second control signal to a temperature control device 840 in the scanning cabin, and the temperature control device 840 may keep the animal warm based on the second control signal.

In some embodiments, the temperature control device 840 may be located in the scanning cabin, and when the scanning cabin is disposed on the scanning cabin management module or the imaging module, the temperature control device 840 may detect the actual temperature in the scanning cabin and keep the animal warm in the scanning cabin based on the control instruction of the control module 810, the control instruction of the imaging module 820, or the control instruction of the scanning cabin management module, respectively. In some embodiments, the temperature control device 840 may include the heating pipeline. In some embodiments, one end of the scanning cabin may be opened with the heating inlet and the heating outlet in communication with the inner cavity of the scanning cabin, and the heating pipeline may be connected to the heating inlet and the heating outlet, respectively. In some embodiments, hot gas or hot water may be passed into the heating pipeline, and the hot gas or hot water may be transmitted to the inner cavity of the scanning cabin through the heating inlet to keep the animal warm. In some embodiments, when the hot water is in the heating pipeline, the scanning cabin may be provided with an accommodating cavity or an accommodating pipe for the hot water passing through. In some embodiments, the hot air/water in the heating pipeline may be discharged from the scanning cabin to cool the animal. By discharging the hot air/water from the heating pipeline into or out of the scanning cabin, the temperature in the scanning cabin, the body temperature of the animal, or the temperature of the heating device may be adjusted.

In some embodiments, the temperature control device 840 may include a heating pad that is provided directly in the scanning cabin for adjusting the temperature of the scanning cabin. There are a variety of manners to heat animal or keeps the animal warm, which are not described herein.

Specifically, since the experimental operation process is to be automated, the temperature in the inner cavity of the scanning cabin should be real-time monitored and electrically adjustable. In some embodiments, the second control signal may include the target temperature, and the temperature control device 840 may adjust the actual temperature of the scanning cabin based on the target temperature.

In some embodiments, the temperature control device 840 may include a temperature detection assembly configured to detect the actual temperature in the temperature control device 840. Since different animals present different vital signs after the anesthesia, the temperature at which the different animals need to be kept warm is different. Therefore, different temperature thresholds may be input to the temperature control device 840 for different situations. In some embodiments, the temperature threshold may be a suitable temperature, e.g., 35° C.-40° C. In some embodiments, the actual temperature detected by the temperature detection assembly in the temperature control device 840 may be compared to the temperature threshold, and when the actual temperature exceeds the temperature threshold, i.e., when the actual temperature exceeds the suitable temperature, the temperature detection assembly may feed the detection signal back to the temperature control device 840. In some embodiments, the temperature control device 840 may adjust the speed or flow rate of the hot gas/hot water that is input into the scanning cabin by the heating pipeline, to adjust the actual temperature in the temperature control device 840 to be within the temperature threshold. By adjusting the speed or flow rate of the hot gas/hot water that is input into the scanning cabin, the actual temperature in the temperature control device 840 may be electrically controlled and adjusted, and the temperature in the scanning cabin may be prevented from being too high or too low due to that the actual temperature in the temperature control device 840 exceeds a temperature threshold, thereby affecting the vital signs of animals.

In some embodiments, a temperature detection assembly may be provided within a scanning cabin (i.e., a scanning cabin) to detect the actual temperature in the scanning cabin. In some embodiments, the actual temperature in the scanning cabin detected by the temperature detection assembly may be compared to a temperature threshold, and when the actual temperature exceeds the temperature threshold, i.e., when the actual temperature exceeds a suitable temperature (e.g., 35° C.-40° C.), the temperature detection assembly may feed the detection signal back to the temperature control device 840. In some embodiments, the temperature control device 840 may adjust the speed or flow rate of the hot gas/hot water that is input into the scanning cabin by the heating pipeline, to adjust the actual temperature of the temperature control device 840 to be within the preset temperature threshold. By adjusting the speed or flow rate of the hot gas/hot water that is input to the scanning cabin, the control of the scanning cabin may be adjusted to avoid the actual temperature of the scanning cabin exceeding the temperature threshold and resulting in the temperature of the scanning cabin being too high or too low, which may affect the vital signs of the animal and the final experimental result.

In some embodiments, the temperature detection assembly may detect the body temperature of the animal. Since different species of animals do not correspond to exactly the same value of suitable body temperature range. Therefore, different body temperature thresholds may be input into the temperature control device 840 for different species of animals. In some embodiments, the body temperature of the animal detected by the temperature detection assembly may be compared to the body temperature threshold, and when the body temperature exceeds the body temperature threshold, i.e., when the body temperature exceeds the suitable body temperature (e.g., 36° C.-37° C.), the temperature detection assembly may feed the detection signal back to the temperature control device 840. In some embodiments, the temperature control device 840 may adjust the speed or flow rate of the hot air/hot water of the scanning cabin that is input by the heating pipeline, to adjust the body temperature of the animal to be within the body temperature threshold. By adjusting the speed or flow rate of the hot air/hot water that is input to the scanning cabin, the body temperature of the animal may be controlled to avoid the body temperature of the animal from being too high or too low, which may affect the vital signs of the animal and the final experimental result.

In some embodiments, the temperature control device 840 and the anesthesia device 830 may be activated at the same time, or the temperature control device 840 may be activated first and then the anesthesia device 830 may be activated, and an order of activation of the temperature control device 840 and the anesthesia device 830 may be adjusted according to the actual situation, which will not be limited herein. For example, when an environment temperature is low, the temperature control device 840 may be activated first and then the anesthesia device 830 may be activated. As another example, when the environment temperature is high, the anesthesia device 830 may be activated first and then the temperature control device 840 may be activated. As another example, when the environment temperature is suitable, the temperature control device 840 and the anesthesia device 830 may be activated simultaneously. In some embodiments, the temperature control device 840 and the aesthesia device 830 are centralizedly controlled through the control module. For example, the control parameter, such as the temperature parameter, the concentration or dosage of anesthesia gas, and other control parameters may be set at the terminal device first, and then centralizedly controlled at the terminal device. The terminal device may be a local terminal device and/or a mobile device.

In some embodiments, after the animal is anesthetized and warmed, the temperature control device 840 may feed the temperature signal back to the control module 810, the temperature signal characterizing the warmed condition of the scanning cabin. In some embodiments, after the control module 810 receives the temperature signal fed back by the temperature control device 840 after completing the heat preservation, the control module 810 may send a sixth control signal to the imaging module 820, and the imaging module 820 may perform the imaging on the animal based on the sixth control signal. In some embodiments, after receiving the temperature signal fed back by the temperature control device 840 after completing the heat preservation, the control module 810 may send the temperature signal to the terminal device (e.g., the one or more terminals 130 or the display screen 315), and the operator may remotely control the imaging module 820 to perform the imaging on the animal.

In some embodiments, when the scanning cabin is located on the scanning cabin management module, the temperature detection assembly may transmit the temperature signal to the scanning cabin management module or the control module 810 through the scanning adapter and the docking connector (not shown in FIG. 8.). In some embodiments, when the scanning cabin is located on the imaging module, the temperature detection assembly may transmit the temperature signal to the imaging module or the control module 810 through a connector and a docking connector. In some embodiments, the control module 810 may receive the temperature signal and send a second control signal to the temperature control device 840 of the scanning cabin, and the temperature control device 840 may keep the animal warm based on the second control signal.

In some specific cases, it is also necessary to inject the animal with a medicine, e.g., a contrast agent, to improve the contrast degree of a specific part of the image. That is, after the animal is anesthesia and warmed, the animal also needs to be injected to perform the scanning on the animal in the scanning cabin through the imaging module 820. In some embodiments, the animal may be injected with the medicine in the scanning cabin management module, or the animal may be injected with the medicine in the imaging module.

Further, in some embodiments, the imaging system 800 may further include an injection device 850. In some embodiments, the control module 810 and the injection device 850 may be electrically connected. In some embodiments, after the injection device 850 receives the third control signal sent by the control module 810, the injection device 850 may inject the animal based on the third control signal and feed the injection signal back to the control module 810, and the injection signal may characterize the injection condition of the animal. In some embodiments, the injection device 850, the temperature control device 840, and the anesthesia device 830 may be all centralizedly controlled through the control module. In some embodiments, the control module 810 may receive the injection signal and send a third control signal to control the injection device 850 to perform a corresponding action. For example, the temperature parameter, the concentration parameter of anesthesia gas or dosage, the injection dosage parameter, the injection time parameter, and other control parameters may be set at the terminal device first, and then centrally controlled at the terminal device. The terminal device may be a local terminal device and/or a mobile device.

Specifically, in some embodiments, the injection device 850 may include a high-precision injection pump. The high-precision injection pump may be electrically adjustable for injection speed and injection dosage when injecting the animal. In some embodiments, the third control signal may include the injection parameter. In some embodiments, the injection parameter may include at least one of the injection dosage parameter, the injection speed parameter, or an injection time parameter. In some embodiments, the injection time parameter may include an injection start moment and an injection end moment. In some embodiments, the injection device 850 may perform the medicine injection on the animal in the scanning cabin based on the at least one of the injection dosage parameter, the injection speed parameter, or the injection time parameter included in the third control signal. In some embodiments, when performing an animal scanning experiment, after performing a preliminary experimental preparation, the experimenter selects the scanning protocol on the operation device, and after the system is activated, the control device may send the preset setting value in the scanning protocol to the anesthesia device 830, the temperature control device 840, and the injection device 850, respectively, to start an initiation of the inhalation anesthesia, the body temperature heat preservation, and the medicine injection, and automatically synchronize the medicine injection information the experiment registration information to the experiment registration information, which reduces the complexity of the entire a scanning operation process, improves the repeatability and precision of the experiments, and is more friendly to the operator. The control module 810 may begin to activate the imaging module 820 to perform the scanning on the animal according to a post-injection scanning start time.

In some embodiments, the injection device 850 may include an injection dosage detection assembly configured to detect an actual injection dosage in the injection device 850. Since the amount of injection dosage to be used is often different for different species of animals and different states of the same animal. Therefore, different thresholds of the injection dosage may be input to the injection device 850 for different situations. In some embodiments, the injection dosage threshold may be a suitable injection dosage, e.g., I-2 mmol/kg. In some embodiments, the injection dosage detected by the injection dosage detection assembly may be compared to the injection dosage threshold, and when the injection dosage exceeds the injection dosage threshold, that is, when the injection dosage exceeds the suitable injection dosage, the injection dosage detection assembly may feed the detection signal back to the injection device 850. In some embodiments, the injection device 850 may adjust the actual injection dosage to be within the injection dosage threshold by adjusting the injection dosage at the time of performing the medicine injection on the animal, thereby realizing an electrical controllable adjustability of the injection dosage in the injection device 850, and avoiding that the actual injection dosage in the injection device 850 exceeds an injection dosage threshold and causing injury to the animal, or the injection speed is so slow that the imaging effect is affected.

In some embodiments, the injection device 850 may include an injection speed detection assembly configured to detect an actual injection speed in the injection device 850. Since different species of animals and different states of the same animal often require different injection speeds to be used. Therefore, different thresholds of the injection speed may be input to the injection device 850 for different situations. In some embodiments, the injection speed threshold may be a suitable injection speed, for example, 1-2 ml/s. In some embodiments, the actual injection speed of the injection device 850 detected by the injection speed detection assembly may be compared to the injection speed threshold, and when the actual injection speed exceeds the injection speed threshold, that is, when the actual injection speed exceeds the suitable injection speed, the injection speed detection assembly may feed the detection signal back to the injection device 850. In some embodiments, the injection device 850 may adjust the actual injection speed to be within the injection speed threshold by adjusting the actual injection speed at the time of performing the medicine injection on the animal, thereby realizing an electrical controllable adjustability of the injection dosage in the injection device 850, and avoiding that the injection dosage in the injection device 850 exceeds the injection dosage threshold and causing injury to the animal, or the injection speed is so slow that the dosage requirement is not satisfied.

In some embodiments, after the injection device 850 completes injecting the animal, the injection device 850 may feed the injection signal back to the control module 810 or the imaging module 840, and the injection signal may characterize an injection condition of the animal. In some embodiments, after receiving the injection signal fed back by the injection device 850 after completing the injection, the control module 810 may again send the sixth control signal to the imaging module 820 that performs the scanning (e.g., a magnetic resonance scan) on the animal based on the sixth control signal. In some embodiments, after receiving the injection signal fed back by the injection device 850 after completing the injection, the control module 810 may send the sixth control signal to a terminal device (e.g., the one or more terminals 130 or the display screen 315) for an operator to remotely control the imaging module 820 to perform the scanning on the animal.

In some embodiments, the injection device 850, the temperature control device 840, and the anesthesia device 830 may be all centralizedly controlled through the control module, and the injection device 850 is used in conjunction with the physiological monitoring device, the temperature control device 840, and the anesthesia device 830, which prevents the animal from awakening during the manual injection process, keeps the animal in a suitable life state, and improves the experimental efficiency.

In some embodiments, the injection device 850 may complete an injection of an animal in the scanning cabin and may feed the injection signal back to the control module 810, the injection signal characterizing an injection condition of the animal. In some embodiments, the control module 810 may send the fourth control signal to the physiological monitoring device to obtain a physiological parameter signal of the animal after receiving the injection signal fed back by the injection device 850 after completing the injection. In some embodiments, after the control module 810 receives the physiological parameter signal, in response to determining that the physiological parameter is abnormal (e.g., the heartbeat rate is too high or too low), the control module 810 may then send the second control signal to the temperature control device 840, and the temperature control device 840 may keep the animal warm based on the second control signal.

In some embodiments, when the scanning cabin is located on the imaging module, the injection dosage detection assembly or the injection speed detection assembly may transmit the injection signal to the imaging module or the control module 810 through the connector and the docking connector.

In some embodiments, physiological parameter monitoring of the animal may be performed in the scanning cabin management module and the imaging module, respectively. In some embodiments, the imaging system 800 may include a physiological monitoring device (not shown in FIG. 8). In some embodiments, the control module 810 may be electrically connected to the physiological monitoring device. In some embodiments, after the physiological monitoring device receives the fourth control signal sent by the control module 810, the physiological monitoring device may perform physiological monitoring on the physiological parameter of the animal based on the fourth control signal, and feed the physiological parameter signal back to the control module 810, and the physiological parameter signal may characterize a physiological condition of the animal, such as at least one of the number of heartbeats, the heartbeat rate, the respiratory signal, the body temperature signal, and/or the blood oxygen signal. In some embodiments, the control module 810 may receive the physiological parameter signal and send the fourth control signal to control a relevant device of the imaging system 800 (e.g., the injection device, the temperature control device 840) to perform a corresponding action. In some embodiments, when the scanning cabin is located on the imaging module, the physiological monitoring device may transmit the physiological parameter signal to the imaging module 820 or the control module 810 through the connector and the docking connector.

In some embodiments, the physiological monitoring device may include a heartbeat monitoring assembly configured to monitor the heartbeat rate of the animal. In some embodiments, the heartbeat monitoring assembly may monitor the heartbeat rate of the animal continuously, or may monitor the heartbeat rate of the animal at intervals (e.g., every 3 minutes or 5 minutes of detection). Since different species of animals and different states of the same animal often correspond to different heartbeat rates. Therefore, different heartbeat rate thresholds may be input to the physiological monitoring device for different situations. In some embodiments, the heartbeat rates for different species of animals and the heartbeat rates for the same species of animals in different states (e.g., different weights, different ages) in a normal state of vital signs may be obtained after big data analysis. In some embodiments, based on the heartbeat rate, the experimenter may input a heartbeat rate threshold to the physiological monitoring device, which is a heartbeat rate of the animal being in a normal state of vital signs. In some embodiments, the heartbeat rate threshold may be a suitable heartbeat rate, e.g., a normal heartbeat rate range of 328-780 beats/minute for mice. In some embodiments, the heartbeat rate of the animal monitored by the heartbeat monitoring assembly may be compared to the heartbeat rate threshold, and when the heartbeat rate of the animal exceeds the heartbeat rate threshold, that is, when the heartbeat rate of the animal exceeds a heartbeat rate when the animal is in the normal state of vital signs, the heartbeat monitoring assembly may feed the heartbeat rate monitoring signal back to the control module 810. In some embodiments, the control module 810 may send a control instruction (e.g., an abnormal reminder signal) to a voice device or a reminder device (not shown in FIG. 8), and the control instruction may cause the voice device or the reminder device to send a reminder message to a terminal device associated with the experimenter. The reminder message may alert the experimenter to take measures as soon as possible. For example, the voice device may emit a beep tone, an alarm tone, to remind the experimenter to stop scanning or anesthetizing the animal again. As another example, the reminder device may emit a text reminder message to remind the experimenter to stop scanning or anesthetizing the scanning cabin. By monitoring the heartbeat rate of the animal and taking corresponding measures when the heartbeat rate exceeds the heartbeat rate threshold, the heartbeat rate of the animal in the scanning cabin exceeds the heartbeat rate threshold and affects the final scanning result.

The number of heartbeats, the respiratory signal, and the heartbeat rate may be similar, and the descriptions of monitoring of the number of heartbeats, the respiratory signal, and controlling other devices in the imaging system 800 (e.g., a voice device or an injection device 850) to perform a corresponding operation based on the monitoring signal may be found in the above description of monitoring the heartbeat rate and performing a corresponding operation by other devices in the imaging system 800 when the heartbeat rate exceeds the heartbeat rate threshold, which will not be repeated herein.

In some embodiments, the physiological monitoring device may include an oximetry monitoring assembly configured to monitor the blood oxygen of the animal. In some embodiments, the oximetry monitoring assembly may continuously monitor a blood oxygen index of the animal or may monitor the blood oxygen index of the animal at intervals (e.g., every 3 minutes or 5 minutes of detecting). In some embodiments, the blood oxygen index may include an oxygen partial pressure, an oxygen content, an oxygen capacity, an oxygen saturation, and a difference in oxygen content between arterial and venous blood. Since different species of animals and different states of the same animal often correspond to different blood oxygen index. Therefore, different thresholds of the blood oxygen index may be input to the physiological monitoring device for different conditions. In some embodiments, the blood oxygen threshold may include a threshold of the blood oxygen partial pressure, a threshold of the blood oxygen content, a threshold of the blood oxygen capacity, a threshold of the blood oxygen saturation, and a threshold of the arterial-venous blood oxygen content. In some embodiments, the blood oxygen for different species of animals, and the same species of animals in different states (e.g., different weights, different ages) in the normal state of vital signs may be obtained after the big data analysis. In some embodiments, based on the blood oxygen index, the experimenter may input a blood oxygen threshold, that is, the blood oxygen index of the animal in the normal state of vital signs, in the physiological monitoring device. In some embodiments, the blood oxygen threshold may be suitable for the blood oxygen index, e.g., the blood oxygen partial pressure of the mouse may be 80-110 mm Hg. In some embodiments, the blood oxygen index of the animal monitored by the blood oxygen monitoring assembly may be compared to a corresponding blood oxygen threshold, and when the blood oxygen index of the animal exceeds the blood oxygen threshold, that is, when the animal is in a normal state of vital signs, the experimenter may input the blood oxygen index to the physiological monitoring device. When the blood oxygen index of the animal exceeds the blood oxygen threshold value, that is, when the blood oxygen index of the animal exceeds the corresponding blood oxygen index when the animal is in the normal state of vital signs, the blood oxygen monitoring assembly may feed the blood oxygen signal back to the control module 810. In some embodiments, the control module 810 may send a control instruction (e.g., an abnormal reminder signal) to the voice device or the reminder device, and the control instruction may cause the voice device or the reminder device to send a reminder message to a terminal device associated with the experimenter. The reminder message may alert the experimenter to take measures as soon as possible. For example, the voice device may emit a beep tone, an alert tone, to remind the experimenter to stop scanning or anesthetizing the animal again. As another example, the reminder device may emit a reminder message to remind the experimenter to stop scanning or anesthetizing the animal again.

In some embodiments, the physiological monitoring device may include a body temperature monitoring assembly configured to monitor the body temperature of the animal. In some embodiments, the body temperature monitoring assembly may continuously monitor the body temperature of the animal or may monitor the body temperature of the animal at intervals (e.g., every 3 minutes or 5 minutes of detection). The related descriptions of the body temperature and controlling the temperature control device 840 to perform the corresponding operation based on the body temperature may be found in the descriptions of other parts in the present disclosure, which will not be repeated herein. In some embodiments, when the body temperature of the animal exceeds a body temperature threshold, i.e., when the body temperature of the animal exceeds a suitable body temperature corresponding to the animal in the normal state of vital signs, the body temperature monitoring assembly may feed the body temperature signal back to the control module 810. In some embodiments, the control module 810 may send a control instruction (e.g., an abnormal reminder signal) to the voice device or the reminder device, and the control instruction may cause the voice device or the reminder device to send the reminder message to the terminal device associated with the experimenter. The reminder message may alert the experimenter to take measures as soon as possible. For example, the voice device may emit the beep tone, and alarm tone, to remind the experimenter to stop scanning or warming the scanning cabin. As another example, the reminder device may emit a textual reminder message to remind the experimenter to stop scanning or warming the scanning cabin.

In some embodiments, in response to determining that the heartbeat rate of the animal is within the heartbeat rate threshold, the blood oxygen index may be within the blood oxygen threshold, or the body temperature may be within the body temperature threshold. The control module 810, after receiving the heartbeat signal, the blood oxygen signal, or the temperature signal, may send a sixth control signal to the imaging module 820, and the imaging module 820 may scan the animal based on the sixth control signal (e.g., a magnetic resonance scan). In some embodiments, in response to determining that the heartbeat rate of the animal is within the heartbeat rate threshold, the blood oxygen index is within the blood oxygen threshold, or the body temperature is within the body temperature threshold, the control module 810, after receiving a heartbeat rate signal, the blood oxygen signal, or the temperature signal, may send the heartbeat rate signal, blood oxygen signal, or temperature signal to a terminal device (e.g., the one or more terminals 130 or the display screen 315), and the operator may remotely control the imaging module 820 to perform the scanning (e.g., an MRI scan) on the animal.

In some embodiments, the physiological parameter monitoring may include a heartbeat signal, and the imaging module 820 may be controlled to scan the animal at a fixed point in time for each heartbeat signal cycle, or known as a cardiac cycle. In some embodiments, the physiological parameter monitoring may include a respiratory signal that controls the imaging module 820 to scan the animal at a fixed time point of each respiratory cycle.

In some embodiments, operations, such as inputting the anesthesia gas to the scanning cabin, adjusting the actual temperature in the scanning cabin or the body temperature of the subject, injection of the subject, and monitoring the physiological, may be performed simultaneously or sequentially. In some embodiments, in response to determining that the operations of simultaneously or sequentially inputting the anesthesia gas to the scanning cabin, adjusting the temperature of the scanning cabin or the body temperature of the subject, scanning the subject, and monitoring the physiological parameters are performed, and the physiological parameter is in the threshold range, the control module 810 may, based on the signals of completion of the above operations and normalization of the physiological parameters, send the sixth signal to the imaging module 820, to control the imaging module 820 to perform the imaging on the animal.

In some embodiments, the control module 810 may include a mobile device (not shown in FIG. 8), and the other devices in the imaging system 800 (e.g., the imaging module 820, the anesthesia device 830, the temperature control device 840, the injection device 850, or the physiological monitoring device) are connected to the mobile device through the wireless communication. By disposing the control module 810 into the mobile device, the experimenter may remotely control and monitor the scanning cabin through the mobile device during the experiment, the experimenter does not necessarily to stay in the lab to wait for the completion of the experiment, which increases the convenience of the experimental process.

The imaging system 800 provided by the embodiments of the present disclosure embodiments, by linking the control module 810 with the imaging module 820, the anesthesia device 830, the temperature control device 840, the injection device 850, or the physiological monitoring device, may make the anesthesia, the heat preservation, the injection, and the scanning of the animal fully automated, and the intermediate process does not require the participation of the operator, reduces the complexity of the operation process, which reduces the complexity of the entire a scanning operation process, improves the repeatability and precision of the experiments, and is more friendly to the operator. Moreover, the control module 810 begins to activate the imaging module 820 to scan the animal according to the post-injection scanning start time. Moreover, the anesthesia gas parameter data, the temperature parameter data, the injection parameter data, and the physiological parameter data during the experiment are automatically synchronized to the experimental registration information, which facilitates directly citing the parameters in the database for the next experiment, further simplifying the experimental process. In addition, the experimenter may remotely control the scanning cabin by disposing the control module 810 into the mobile device and connecting the other devices of the imaging system 800 with the mobile device through wireless communication, such that the experimenter does not necessarily to stay in the lab to wait for the completion of the experiment, which increases the convenience of the experimental process.

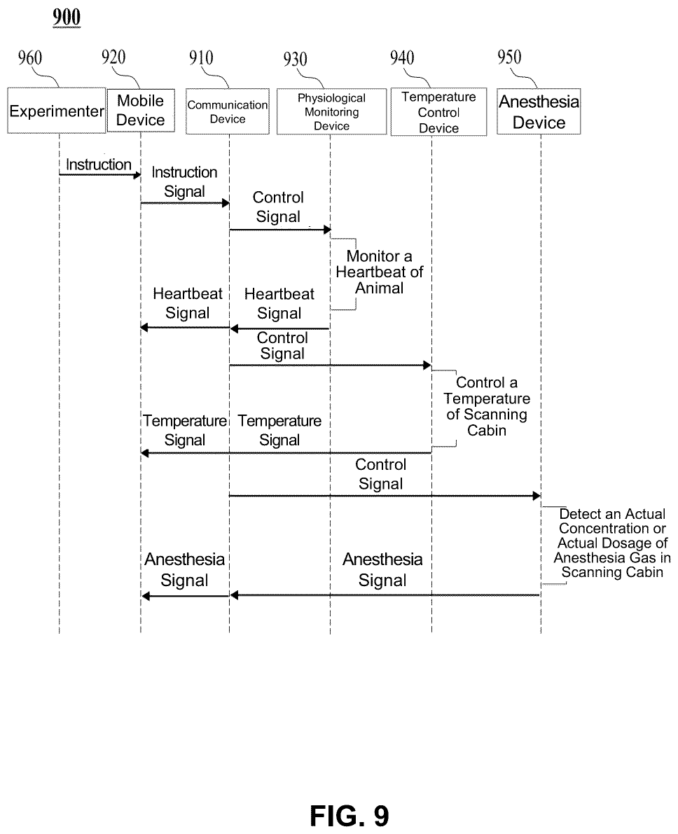

FIG. 9 is a flowchart illustrating an exemplary imaging system 900 according to another embodiment of the present disclosure.

Referring to FIG. 1, in some embodiments, the imaging system 900 may be applied to in a scanning cabin, where the scanning cabin may be typically placed in an imaging module or an imaging device to perform a magnetic resonance imaging, and the imaging module or the imaging device may be placed in a scanning room of magnetic resonance imaging to perform the imaging on the animal.

When performing an in vivo scanning on the animal, the image or result required may often require a long period of time. During the MRI scanning, the state of the animal needs to be monitored in real-time, and the experimenter 960 may usually need to stay in the scanning room for a long period of time to wait for the completion of the scanning, which is time-consuming and laborious when performing the scanning with such a long period of time (e.g., more than 10 hours).

The embodiments of the present disclosure provide an imaging system 900. In some embodiments, the imaging system 900 may include a communication device 910, a physiological monitoring device 930, a temperature control device 940, and an anesthesia device 950. The communication device 910 may be connected to the mobile device 920 through the wireless communication, and the communication device 910 may be electrically connected to the function assistance modules, such as the physiological monitoring device 930, the temperature control device 940, the anesthesia device 950, a scanning cabin management module and an imaging module, respectively. In some embodiments, the imaging system 900 may also include a local terminal device (not shown in FIG. 9), and the communication device 910 may be connected to the local terminal device through the wired or wireless communication, and the local terminal device may control other components of the imaging system 900. By disposing the mobile device 920 and the local terminal device, the experimenter 960 may locally control the other components of the imaging system 900 through the local terminal device, and may remotely obtain a monitoring parameter (e.g., the heartbeat information, the temperature parameter, and the anesthesia parameter, etc.) in real-time and remotely control the scanning system 900 through the mobile device 920.

In some embodiments, the imaging system 900 may further include an injection device (not shown in FIG. 9), and the communication device 910 may be electrically connected to the injection device. In some embodiments, the mobile device 920 may receive an instruction from the experimenter 960 and send an instruction signal to the communication device 910. In some embodiments, the communication device 910 may send control signals (e.g., the first control signal, the second control signal, etc.) to the physiological monitoring device 930, the temperature control device 940, the anesthesia device 950, and the injection device based on the instruction signal. In some embodiments, the physiological monitoring device 930 may feed the physiological parameter signal back to the mobile device 920 through the communication device 910. In some embodiments, the temperature control device 940 may feed the temperature signal back to the mobile device 920 through the communication device 910. In some embodiments, the anesthesia device 950 may feed the anesthesia signal back to mobile device 920 through communication device 910. In some embodiments, the injection device may feed the injection signal back to the mobile device 920 through the communication device 910. In some embodiments, the experimenter 960 may remotely obtain the above signals in real-time through the mobile device 920 and remotely adjust the above components of the imaging system 900 based on the above control signals. It should be noted that in the present disclosure, a parameter included in a control signal (e.g., the first control signal, the second control signal, the third control signal, etc.) or a control instruction generated by a control module or control device may include a reference value or range, a target value or range, or a desired value or range of the parameter; a parameter included in a feedback signal (e.g., a physiological parameter signal, the temperature signal, the anesthesia signal, etc.) generated by a monitoring device (e.g., the physiological monitoring device, the temperature control device, the anesthesia device, etc.) may include an actual value of the parameter. For example, the temperature parameter included in the second control signal may include a target temperature of the scanning cabin or a target body temperature of the subject, and the temperature parameter of a temperature signal feed back to the control module refers to an actual value of the temperature, e.g., an actual value of the temperature of the scanning cabin, an actual value of the temperature of the subject, an actual value of the temperature of the heating device, etc., that may be detected by a temperature detection assembly of a temperature control device or module. As another example, the anesthesia parameter included in the first control signal may include a target value of the anesthesia parameter of the subject, and the anesthesia parameter of an anesthesia signal feed back to the control module refers to an actual value of the anesthesia parameter that may be detected by the anesthesia device or module. As still another example, the injection parameter included in the third control signal may include a target value of the injection parameter of the subject, and the injection parameter of an injection signal feed back to the control module refers to an actual value of the injection parameter that may be detected by the injection device or module. As still another example, the physiological parameter included in the fourth control signal may include a target value of the physiological parameter of the subject, and the physiological parameter of an physiological parameter signal feed back to the control module refers to an actual value of the physiological parameter that may be detected by the physiological monitoring device or module.

It should be noted that, in the embodiments of the present disclosure, by connecting the communication device 910 with the mobile device 920 through the wireless communication, and electrically connecting the communication device 910 with the physiological monitoring device 930, the temperature control device 940, and the anesthesia device 950, respectively, the experimenter may obtain the monitoring parameter in real-time through the mobile device 920 with a long period of time, thereby remotely controlling the imaging system 900, such that the experimenter is not necessary to stay in the lab to wait for the completion of the experiment, which increases the convenience of the experimental process.

It should be noted that, the physiological monitoring device 930, the temperature control device 940, and the anesthesia device 950 are disposed in a scanning room of the magnetic resonance imaging lab, and the communication device 910 and an operating table may be provided in the operation room, to be able to perform a local operation and a local control of a device or a module in the scanning room within the operation room. In some embodiments, the operation room and the scanning room may be separated from each other, and the communication device 910 may be electrically connected to the physiological monitoring device 930, the temperature control device 940, and the anesthesia device 950, respectively, that is, the communication device 910 is connected to the physiological monitoring device 930, the temperature control device 940, and the anesthesia device 950, respectively through the cables, after the cables are powered on, the communication device may be powered on with the physiological monitoring device 930, the temperature control device 940, and the anesthesia device 950, respectively, thereby realizing an electrical connection. Through the above setting, the communication device 910 may directly perform a local control on the physiological monitoring device 930, the temperature control device 940, and the anesthesia device 950, respectively, through the cables.

In some embodiments, the mobile device 920 may be a removable control panel, e.g., a cell phone, a tablet, a computer, etc., and have a software operating system and its functionality on the mobile device 920 to remotely control the communication device 910, the physiological monitoring device 930, the temperature control device 940, and the anesthesia device 950 through the mobile device 920.

Further, to understand changes in the life condition of the animal in the scanning cabin, then it is necessary to perform a real-time physiological monitoring on the animal (e.g., a heartbeat condition, a respiration condition, a body temperature condition, a blood oxygen condition). In some embodiments, the physiological parameter signal that the physiological monitoring device 930 feeds back to the communication device 910 may include at least one of the numbers of heartbeats, the heartbeat rate, the respiratory signal, the body temperature signal, and the blood oxygen signal, and the vital signs of the animal may be determined by the numbers of heartbeats, the heartbeat rate, the respiratory signal, the body temperature signal, and the blood oxygen signal of the animal.

Specifically, in some embodiments, to perform the real-time monitoring on the heartbeat of the animal, the physiological monitoring device 930 may be provided with a heartbeat monitoring assembly, and the heartbeat monitoring assembly may be configured to monitor the heartbeat rate of the animal. In some embodiments, a real-time heartbeat rate of the animal monitored by the heartbeat monitoring assembly may be compared to the heartbeat rate threshold, and when the heartbeat rate of the animal exceeds the heartbeat rate threshold, that is, when the heartbeat rate of the animal exceeds a heartbeat rate when the animal is in the normal state of vital signs, the heartbeat monitoring assembly may feed a heartbeat rate monitoring signal back to the communication device 910. In some embodiment, the communication device 910 may transmit the heartbeat rate monitoring signal to the mobile device 920, and the experimenter 960 may be informed of the heartbeat rate of the animal at a first time through the mobile device 920. In some embodiments, based on the heartbeat rate monitoring signal, the mobile device 920 may send a corresponding reminder signal to facilitate the experimenter 960 to take corresponding measures. Specifically, in some embodiments, when the heartbeat rate exceeds the heartbeat rate threshold, the mobile device 920 may send a voice to remind the experimenter 960 to take measures as soon as possible. For example, the mobile device 920 may emit the beep tone and the alarm tone to remind the experimenter to stop scanning or anesthetizing the animal again. In some embodiments, based on the heartbeat rate monitoring signal, the experimenter 960 may send the control signal, through the mobile device 920, to other devices of the imaging system 900 (e.g., anesthesia devices) to cause other devices of the imaging system 900 to perform corresponding operations. Specifically, in some embodiments, when the heartbeat rate exceeds the heartbeat rate threshold, the experimenter 960 may send an instruction signal to the communication device 910 through the mobile device 920 and send a first control signal to the anesthesia device 950 through the communication device 910 to anesthetize the animal again. By monitoring the heartbeat rate of the animal and taking corresponding linkage measures when the heartbeat rate exceeds the heartbeat rate threshold, it may be avoid that the heartbeat rate of the animal in the scanning cabin exceeds the heartbeat rate threshold and affects the results of the final experiment.

Further, in some embodiments, the heartbeat monitoring assembly may include interconnected ECG lead wires. In some embodiments, one end of the cardiac lead wires may be connected to a display screen of the scanning cabin management module, and the other end of the cardiac lead wires may be connected to a heart position of the animal to monitor the heartbeat rate of the animal and display it on the display screen. By connecting the electrocardiographic lead wires to the heart position of the animal, the heartbeat rate of the animal may be monitored, thereby monitoring the vital signs of the animal in real-time. Display of the heartbeat rate of the animal on the display screen may facilitate real-time reading of the heartbeat rate of the animal by the experimenter 960 located in the operation room. In some embodiments, the heartbeat rate of the animal may be fed back to the communication device 910 through the physiological monitoring device 930 as a heartbeat signal (also referred to as the “heartbeat rate monitoring signal”), and the communication device, in turn, may transmit the heartbeat signal to the local terminal device and/or the mobile device 920, so that the experimenter 960 may read the heartbeat rate of the animal on the local terminal device in real-time. The experimenter 960 may read the heartbeat rate information of the animal in real-time on the local terminal device, and the experimenter 960, who is not in the lab, may also obtain the heartbeat rate information of the animal through the mobile device 920.

In some embodiments, to perform a real-time monitoring on the body temperature and/or blood oxygen of an animal, a body temperature monitoring assembly and/or a blood oxygen monitoring assembly may be configured to be provided in the physiological monitoring device 930, the body temperature monitoring assembly may monitor the body temperature of the animal, and the blood oxygen monitoring assembly may monitor the blood oxygen index of the animal.

In some embodiments, the body temperature of the animal monitored by the body temperature monitoring assembly may be compared to the body temperature threshold, and when the body temperature of the animal exceeds the body temperature threshold, that is, when the body temperature of the animal exceeds a body temperature of the animal in the normal state of vital signs, the body temperature monitoring assembly may feed the body temperature signal back to the communication device 910. In some embodiments, the communication device 910 may transmit the body temperature signal to the mobile device 920, and the experimenter 960 may first learn the body temperature state of the animal through the mobile device 920.

In some embodiments, the blood oxygen index of the animal monitored by the blood oxygen monitoring assembly may be compared to the blood oxygen threshold, and when the blood oxygen index of the animal exceeds the corresponding blood oxygen threshold, that is, when the blood oxygen index of the animal exceeds the blood oxygen corresponding to the animal in the normal state of vital signs, the blood oxygen monitoring assembly may feed the blood oxygen signal back to the communication device 910. In some embodiments, the communication device 910 may transmit the blood oxygen signal to the mobile device 920, and the experimenter 960 may be informed of the blood oxygen state of the animal at the first time through the mobile device 920.

In some embodiments, the temperature control device 940 may include a heating pipeline. More descriptions of the temperature control device 940 may be found in the descriptions in other parts of the present disclosure (e.g., the description of FIG. 8), which will not be repeated herein.

Specifically, since the overall scanning experiment operation process should be automated and remotely controlled, the temperature in an inner cavity of the scanning cabin should be real-time monitored and electrically adjustable. In some embodiments, the temperature signal that the temperature control device 940 may feed back to the communication device 910 may include at least one of the temperature in the scanning cabin, the body temperature of the animal, and the temperature of the heating device.

In some embodiments, the temperature control device 940 may include a temperature detection assembly configured to monitor the temperature in the scanning cabin. Since the temperature in the scanning cabin directly affects the body temperature of the animal, and the temperature in the scanning cabin in turn directly affects the life condition of the animal, such that the temperature in the scanning cabin may be adjusted and controlled in real-time. In some embodiments, temperatures, i.e., a temperature thresholds, suitable for different species of animals, and the same species of animals in different states (e.g., different weights, different ages), may be obtained by the big data analysis. In some embodiments, the actual temperature in the temperature control device 940 monitored by the temperature detection assembly may be compared to the temperature threshold, and when the actual temperature exceeds the temperature threshold, i.e., when the actual temperature exceeds a suitable temperature of the scanning cabin, the temperature detection assembly may feed the temperature signal back to the communication device 910. In some embodiments, the communication device 910 may transmit the temperature signal to the mobile device 920. In some embodiments, the experimenter 960 may be configured to obtain the temperature in the scanning cabin through the mobile device 920 and send the instruction signal to the communication device 910. In some embodiments, the communication device 910 may be configured to send the second control signal to the temperature control device 940 based on the instruction signal, the temperature control device 940 may be configured to adjust the speed or flow rate of the hot gas/hot water that is input to scanning cabin through the heating pipeline, to adjust the actual temperature to be within the temperature threshold. Through the above process, the electrical control of the temperature control device 940 and the adjustment of the real-time temperature in the scanning cabin may be realized, which may avoid that the actual temperature in the scanning cabin exceeds the temperature threshold and cause the temperature in the scanning cabin to be too high or too low, thereby affecting the vital signs of the animal and the final results of the experiment.

In some embodiments, the temperature detection assembly may monitor the body temperature of the animal. The related description of monitoring the real-time temperature of the animal by the temperature detection assembly may be found in the relevant description of FIG. 8 and will not be repeated herein. In some embodiments, the body temperature in the temperature control device 940 monitored by the temperature detection assembly may be compared to a body temperature threshold, and when the actual body temperature exceeds the body temperature threshold, i.e., when the actual body temperature exceeds a suitable body temperature of the animal, the temperature detection assembly may feed the body temperature signal back to the communication device 910. In some embodiments, the communication device 910 may be configured to transmit the body temperature signal to the mobile device 920. In some embodiments, the experimenter 960 may obtain the body temperature of the animal through the mobile device 920 and send the instruction signal to the communication device 910. In some embodiments, the communication device 910 may send the second control signal based on the instruction signal to the temperature control device 940, and the temperature control device 940 may adjust the speed or flow rate of the hot gas/hot water input to the scanning cabin through the heating pipeline based on the second control signal, so that the actual body temperature may be adjusted to be within the body temperature threshold. Through the above process, the electrical adjustment of the temperature control device 940 and the adjustment of the body temperature of the animal may be realized, which may avoid the temperature in the scanning cabin from being too high or too low, and affect the vital signs of the animal and the final experimental result.

In some embodiments, the physiological monitoring device 930 and the temperature control device 940 may be activated at the same time, or the temperature control device 940 may be activated first and then the physiological monitoring device 930 may be activated, or the physiological monitoring device 930 may be activated first and then the temperature control device 940 may be activated, which needs to be adapted according to the actual situation, and is not limited herein.

In some embodiments, the concentration of the anesthesia gas or dosage in the inner cavity of the scanning cabin may be monitored and electrically adjustable to realize the automated control of the overall experimental operation process. In some embodiments, the anesthesia device 950 may include an anesthesia detection assembly configured to monitor a concentration dosage of the anesthesia gas in the scanning cabin. More descriptions of the anesthesia device 950 may be found in the descriptions of other parts in the present disclosure (e.g., the description of FIG. 8) and will not be repeated herein.

In some embodiments, the anesthesia signal fed back to the mobile device 920 through the communication device 910 may include at least one of the concentration of the anesthesia gas and the dosage of the anesthesia gas.

In some embodiments, the actual concentration of the anesthesia gas or the actual dosage of the anesthesia gas in the anesthesia device 950 detected by the anesthesia detection assembly may be compared to the anesthesia threshold. More about anesthesia threshold may be found in the descriptions of other parts in the present disclosure (e.g., the description of FIG. 8) and will not be repeated herein. In some embodiments, when the actual concentration of the anesthesia gas or dosage of the anesthesia gas exceeds the anesthesia threshold, the anesthesia detection assembly may feed the anesthesia signal (e.g., a concentration detection signal or a dosage detection signal) back to the communication device 910. In some embodiments, the communication device 910 may transmit the anesthesia signal to the mobile device 920. In some embodiments, the experimenter 960 may obtain a real-time concentration of the anesthesia gas or an actual dosage of the anesthesia gas situation in the scanning cabin through the mobile device 920, and send an instruction signal to the communication device 910. The communication device 910 may send a first control signal to the anesthesia device 950 based on the instruction signal. In some embodiments, the anesthesia device 950 may adjust the actual anesthesia gas by adjusting an output rate or dosage of the anesthesia gas that is output based on the first control signal, to adjust the actual concentration of the anesthesia gas or the dosage of the actual anesthesia gas to be within the anesthesia threshold. Through the above process, the electrical control of the anesthesia device 950 may be adjusted to avoid over-anesthesia or under-anesthesia of the animal due to the concentration of the anesthesia gas or the dosage of the actual anesthesia gas in the scanning cabin exceeding the anesthesia threshold value, which may not only protect life safety of the animal, but also obtain a better final result of the experiment and a better imaging effect.

In some embodiments, the anesthesia device 950 may be used in conjunction with the temperature control device 940. More descriptions of the linking use between the anesthesia device 950 and the temperature control device 940 may be found in the relevant description of FIG. 8, which will not be repeated herein.

In some embodiments, the injection device may include an injection dosage detection assembly and/or an injection speed detection assembly. The injection dosage detection assembly may detect an actual injection dosage in the injection device, and the injection speed detection assembly may detect an actual injection speed in the injection device. More descriptions of the injection device may be found in the descriptions of other parts in the present disclosure (e.g., the description of FIG. 8) and will not be repeated herein.

In some embodiments, the injection signal fed back to the mobile device 920 through the communication device 910 may include at least one of the injection dosage and the actual injection speed.

In some embodiments, the injection dosage may be compared to an injection dosage threshold. More descriptions of the injection dosage threshold may be found in the descriptions of other parts in the present disclosure (e.g., the description of FIG. 8) and will not repeated herein. In some embodiments, when the actual injection dosage exceeds the injection dosage threshold, the injection dosage detection assembly may feed an injection dosage detection signal back to the communication device 910. In some embodiments, the communication device 910 may transmit the injection dosage detection signal to the mobile device 920. In some embodiments, the experimenter 960 may obtain the injection dosage in the scanning cabin through the mobile device 920, and sends the instruction signal to the communication device 910. The communication device 910 may send the third control signal to the injection device based on the instruction signal. In some embodiments, the injection device may adjust the injection dosage to be within the injection dosage threshold by adjusting the injection dosage of the medicine based on the third control signal.

In some embodiments, the actual injection speed may be compared to an injection speed threshold. More descriptions of the injection speed threshold may be found in the descriptions of other parts of the present disclosure (e.g., the description of FIG. 8) and will not be repeated herein. In some embodiments, when the actual injection speed exceeds the injection speed threshold, the injection speed detection assembly may feed an injection speed detection signal back to the communication device 910. In some embodiments, the communication device 910 may transmit the injection speed detection signal to the mobile device 920. In some embodiments, the experimenter 960 may obtain, through the mobile device 920, the actual injection speed in the animal cavity situation and may send the instruction signal to the communication device 910. The communication device 910 may send the third control signal to the injection device based on the instruction signal. In some embodiments, the injection device may adjust the injection speed of the medicine based on the third control signal, to adjust an actual injection speed to be within the injection dosage threshold range.

Through the above process, the adjustment of the injection dosage and the injection speed of the injection device may be realized, to avoid that the injection dosage of the animal exceeds the injection dosage threshold or the actual injection speed exceeds the preset injection speed threshold, which leads to over-dosage or under-dosage of the medicine injected to the animal, which may protect the life safety of the animal and obtain the final experimental result and a better scanning effect.

In some embodiments, the injection device may be used linking with the physiological monitoring device 930, and the temperature control device 940. More descriptions of the linking use between the injection device, the physiological monitoring device 930, and the temperature control device 940 may be found in the relevant description of FIG. 8, which will not be repeated herein.

In some embodiments, the communication device 910 may be connected to the mobile device 920, the physiological monitoring device 930, the temperature control device 940, the anesthesia device 950, and the injection device through the wireless communication, respectively. The present disclosure does not limit the specific connection methods between the communication device 910 and the mobile device 920, the physiological monitoring device 930, the temperature control device 940, the anesthesia device 950, and the injection device.

The imaging system 900 provided in the present disclosure embodiment may realize that the communication device 910 is connected to the mobile device 920 through the wireless communication, and the communication device 910 may be electrically connected to the physiological monitoring device 930, the temperature control device 940, the anesthesia device 950, and the injection device, respectively, such that the experimenter may remotely control the scanning cabin through the mobile device during the experiment, and does not need to wait for a long time for the final completion of the experiment in the lab, which enhances the convenience of the experimental process. Moreover, the linking use between the anesthesia device 950 and the temperature control device 940, and the linking use between the injection device, the physiological monitoring device 930, and the temperature control device 940 may make the anesthesia, heat preservation, injection and scanning of the animal be fully automated, with no need for the operator to participate in the intermediate process, reduces the complexity of the operation process, improves the reproducibility and precision of the experiment, and is more user-friendly for the experimenter.

The beneficial effects brought about by some embodiments of the present disclosure may include, but are not limited to: (1) by placing the subject in the scanning cabin, and then placing a plurality of scanning cabins in a plurality of cabin seats of the scanning cabin management module, and performing anesthesia, heat preservation, and injection operations on the subject before scanning, it may be ensured that the subject is in the state of being scanned, and it is facilitated that the transfer module transfer the scanning cabins of the subject from the scanning cabin management module to the imaging module in a timely manner, which greatly shortens the scanning preparation time of the subject and improves the work efficiency of the scanning cabin management module; and relative to manual handling the scanning cabin, the use of transferring the scanning cabin shortens the transfer time of the scanning cabin and improves the work efficiency of the imaging system; (2) by performing a linking control on the control module with the imaging module, the anesthesia device temperature control device, the injection parameter device and the physiological monitoring device, the anesthesia, heat preservation, injection and scanning of the animal are fully automated with no need for the operator to participate in the intermediate process, which reduces the complexity of the overall operation process, improves the reproducibility and precision of the experiment, and is more user-friendly to the operator; moreover, the data of the anesthesia gas parameter, the data of the temperature parameter, and the data of the injection parameter, and the data of the physiological parameter during the experiment may be automatically synchronized to the experimental registration information, so that it is convenient to directly use the parameters in the database during the next experiment, which further simplifies the experimental process; (3) by connecting the communication device with the mobile device through the wireless communication, and the communication device is electrically connected to the physiological monitoring device, the temperature control device, the anesthesia device and the injection device, respectively, the experimenter may remote control the scanning cabin through the mobile device during experiment, such that the experimenter is not necessary to stay in the lab to wait for the completion of the experiment, which increases the convenience of the experimental process. It should be noted that different embodiments may produce different beneficial effects, and in different embodiments, the beneficial effects may be any one or a combination of any of the above, or any other beneficial effects that may be obtained.

It should be noted that the above embodiments are only used to illustrate the technical solutions of the present invention rather than to limit the technical solutions, and those skilled in the art in the field should understand that those modifications or equivalent replacements to the technical solutions of the present invention without departing from the purpose and scope of the present technical solutions should be covered by the scope of the claims of the present disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

What is claimed is:

1. An imaging system, comprising:
a scanning cabin management module configured to manage at least one scanning cabin, wherein each of the at least one scanning cabin is configured to place a subject;
an imaging module configured to perform a scanning on the subject in the scanning cabin; and
a control module configured to control the scanning cabin management module and/or the imaging module.

2. The imaging system of claim 1, wherein the imaging system further comprises a function assistance module;
the function assistance module includes at least one of: an anesthesia device, a temperature control device, or a physiological monitoring device; wherein
the anesthesia device is configured to receive a first control signal sent by the control module, output an anesthesia gas to the scanning cabin based on the first control signal, and feed an anesthesia signal back to the control module;
the temperature control device is configured to receive a second control signal sent by the control module, adjust a temperature of the scanning cabin based on the second control signal, and feed a temperature signal back to the control module; and
the physiological monitoring device is configured to perform a physiological monitoring on the subject and feed a physiological parameter signal back to the control module.

3. The imaging system of claim 2, wherein the control module is configured to perform a signal transmission and instruction control on at least one of the scanning cabin management module, the imaging module, or the function assistance module through a wireless communication connection.

4. The imaging system of claim 2, wherein
the function assistance module further includes an injection device configured to receive a third control signal sent by the control module, inject the subject based on the third control signal, and feed an injection signal back to the control module;
the control module is connected to at least one of the anesthesia device, the temperature control device, the injection device, or the physiological monitoring device;
the control module is configured to control at least one of the anesthesia device, the temperature control device, the injection device, or the physiological monitoring device to perform a corresponding operation; and/or, to control the imaging module to perform a scanning based on detection information of at least one of the anesthesia device, the temperature control device, the injection device, or the physiological monitoring device.

5. The imaging system of claim 1, wherein the imaging system further comprises a transfer module configured to transfer the scanning cabin from the scanning cabin management module to the imaging module, the control module is connected to a transfer module; and
the control module is configured to control the transfer module to perform a corresponding operation.

6. The imaging system of claim 5, wherein.
the scanning cabin management module is provided with a plurality of cabin seats configured to place a plurality of scanning cabins;
the transfer module is provided between the scanning cabin management module and the imaging module, and is configured to transfer the scanning cabin from the scanning cabin management module to the imaging module.

7. The imaging system of claim 6, wherein the scanning cabin management module includes a mounting base and a rotating substrate, the rotating substrate is rotatably mounted to the mounting base, and the plurality of cabin seats are disposed on the rotating substrate.

8. The imaging system of claim 7, wherein the scanning cabin management module further includes an adapter, the scanning cabin is rotatably mounted to the rotating substrate through the adapter relative to the rotating substrate.

9. The imaging system of claim 7, wherein the scanning cabin management module further includes a display screen configured to display a physiological parameter of the subject, the plurality of cabin seats are distributed on the rotating substrate along a circumferential direction of the display screen.

10. The imaging system of claim 6, wherein the system further comprises a connector that is mounted to the imaging module; and
the transfer module is capable of mounting the scanning cabin to the imaging module through the connector.

11. The imaging system of claim 6, wherein the transfer module includes a displacement unit and a mechanical arm unit, wherein
the displacement unit is able to drive the mechanical arm unit to perform a translation; and
one end of the mechanical arm unit is connected to the displacement unit and the other end of the mechanical arm unit is configured to clamp the scanning cabin.

12. The imaging system of claim 11, wherein the mechanical arm unit includes a clamping assembly and a rotating arm assembly, one end of the rotating arm assembly is connected to the clamping assembly and the other end of the rotating arm assembly is rotatably connected to the displacement unit;
the rotating arm assembly includes a connecting arm and a first rotating motor, the first rotating motor is mounted to the displacement unit, the connecting arm connects the first rotating motor and the clamping assembly, and the first rotating motor is capable of driving the connecting arm to rotate; and
the clamping assembly includes a driving component and a pair of clamping jaws oppositely disposed, the driving component connects the pair of clamping jaws and the rotating arm assembly, the driving component is configured to drive the pair of clamping jaws to rotate relative to the rotating arm assembly, and drive the pair of clamping jaws to be clamped or opened.

13. The imaging system of claim 2, wherein
the subject includes an animal, and the first control signal includes an anesthesia gas parameter;
the anesthesia gas parameter include at least one of a concentration of an anesthesia gas, a dosage of an anesthesia gas, or an output rate of an anesthesia gas, and the anesthesia device outputs an anesthesia gas to the scanning cabin based on the anesthesia gas parameter included in the first control signal.

14. The imaging system of claim 2, wherein the subject includes an animal,
the second control signal includes at least one of a target temperature in the scanning cabin or a target body temperature of the animal; and
the temperature control device adjusts the temperature of the scanning cabin based on the target temperature in the scanning cabin or the target body temperature of the animal.

15. The imaging system of claim 14, wherein the temperature control device includes a heating pad disposed within the scanning cabin; or the
the temperature control device includes a heating pipeline, the heating pipeline is connected to the scanning cabin and transfers hot gas/hot water in the heating pipeline into the scanning cabin.

16. The imaging system of claim 4, wherein
the subject includes an animal, and the third control signal includes an injection parameter; and
the injection parameter includes at least one of an injection dosage, an injection speed, or an injection time, and the injection device injects a medicine to the animal in the scanning cabin based on the injection parameter included in the third control signal.

17. The imaging system of claim 2, wherein the system further comprises a communication device connected to a mobile device through a wireless communication, and/or,
the communication device is connected to a local terminal device through a wired or wireless communication; and
the communication device is electrically connected to the scanning cabin management module, the imaging module, and the function assistance module, respectively.

18. The imaging system of claim 17, wherein the physiological parameter includes at least one of a heart frequency, a respiratory signal, a body temperature signal, or a blood oxygen signal, and the subject is an animal;
the physiological monitoring device is configured to:
monitor the physiological parameter of the animal,
compare the physiological parameter with a preset threshold of the physiological parameter, and
when the physiological parameter exceeds the preset threshold of the physiological parameter, the physiological monitoring device feeds a physiological parameter monitoring signal back to the communication device, and the communication device transmits the physiological parameter monitoring signal to the mobile device.

19. The imaging system of claim 2, wherein the control module centralizedly controls the anesthesia device, the injection device, and the temperature control device.

20. The system of claim 2, wherein
the control module is further configured to compare the received physiological parameter with a preset threshold of the physiological parameter, and
when the physiological parameter exceeds the preset threshold of the physiological parameter, send out an abnormal reminder signal.
